(19) United States
(12) Patent Application Publication
(10) Pub. No.: US 2011/0046191 A1
(43) Pub. Date: Feb. 24, 2011

(54) COMBINATION OF A MUSCARINIC RECEPTOR ANTAGONIST AND A BETA-2-ADRENOCEPTOR AGONIST

(75) Inventors: Finch Harry, Harlow (GB); Wiley Katherine, Loughborough (GB); Dixon JOhn, Loughborough (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignees: ARGENTA DISCOVERY LTD., Harlow, Essex (GB); ASTRAZENECA AB, Sodertalje (SE)

(21) Appl. No.: 12/523,171

(22) PCT Filed: Feb. 6, 2008
(86) PCT No.: PCT/GB08/00404

§ 371 Date: Oct. 26, 2010

(30) Foreign Application Priority Data
Feb. 7, 2007 (GB) 0702385.6

(51) Int. Cl. A61K 31/428; A61P 11/00

(52) U.S. Cl. 514/367

(57) ABSTRACT

The invention provides a pharmaceutical product, kit or composition comprising a first active ingredient which is a selected muscarinic receptor antagonist selected, and a second active ingredient which is a β₂-adrenoceptor agonist, of use in the treatment of respiratory diseases such as chronic obstructive pulmonary disease and asthma.

The present invention relates to combinations of pharmaceutically active substances for use in the treatment of respiratory diseases, especially chronic obstructive pulmonary disease (COPD) and asthma.

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common of the respiratory diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

Therapeutic agents used in the treatment of respiratory diseases include β₂-adrenoceptor agonists. These agents (also known as beta2 (β₂)-agonists) may be used to alleviate symptoms of respiratory diseases by relaxing the bronchial smooth muscles, reducing airway obstruction, reducing lung hyperinflation and decreasing shortness of breath. Compounds currently under evaluation as once-daily β2 agonists are described in Expert Opin. Investig. Drugs 14 (7), 775-783 (2005).

A further class of therapeutic agent used in the treatment of respiratory diseases are muscarinic antagonists. Muscarinic receptors are a G-protein coupled receptor (GPCR) family having five family members M₁, M₂, M₃, M₄ and M₅. Of the five muscarinic subtypes, three (M₁, M₂ and M₃) are known to exert physiological effects on human lung tissue. Parasympathetic nerves are the main pathway for reflex bronchoconstriction in human airways and mediate airway tone by releasing acetylcholine onto muscarinic receptors. Airway tone is increased in patients with respiratory disorders such as asthma and chronic obstructive pulmonary disease (COPD), and for this reason muscarinic receptor antagonists have been developed for use in treating airway diseases. Muscarinic receptor antagonsists, often called anticholinergics in clinical practice, have gained widespread acceptance as a first-line therapy for individuals with COPD, and their use has been extensively reviewed in the literature (e.g. Lee et al, Current Opinion in Pharmacology 2001, 1, 223-229).

Whilst treatment with a β₂-adrenoceptor agonist or a muscarinic antagonist can yield important benefits, the efficacy of these agents is often far from satisfactory. Moreover, in view of the complexity of respiratory diseases such as asthma and COPD, it is unlikely that any one mediator can satisfactorily treat the disease alone. Hence there is a pressing medical need for new therapies against respiratory diseases such as COPD and asthma, in particular for therapies with disease modifying potential.

The present invention provides a pharmaceutical product comprising, in combination, a first active ingredient which is a muscarinic antagonist selected from:
[2-((S)-Cyclohexyl-hydroxy-phenyl-methyl)-oxazol-5-ylmethyl]-dimethyl-(3-phenoxy-propyl)-ammonium salt,
[2-((R)-Cyclohexyl-hydroxy-phenyl-methyl)-oxazol-5-ylmethyl]-dimethyl-(3-phenoxy-propyl)-ammonium salt,
[2-((R)-Cyclohexyl-hydroxy-phenyl-methyl)-oxazol-5-ylmethyl]-dimethyl-(2-phenethyloxy-ethyl)-ammonium salt,
[2-((R)-Cyclohexyl-hydroxy-phenyl-methyl)-oxazol-5-ylmethyl]-[3-(3,4-dichloro-phenoxy)-propyl] dimethyl-ammonium salt,
[2-((R)-Cyclohexyl-hydroxy-phenyl-methyl)-oxazol-5-ylmethyl]-[2-(3,4-dichloro-benzyloxy)-ethyl]-dimethyl-ammonium salt, and
[2-(4-Chloro-benzyloxy)-ethyl]-[2-((R)-Cyclohexyl-hydroxy-phenyl-methyl)-oxazol-5-ylmethyl]-dimethyl-ammonium salt;
   and a second active ingredient which is a β₂-adrenoceptor agonist.

A beneficial therapeutic effect may be observed in the treatment of respiratory diseases if a muscarinic antagonist according to the present invention is used in combination with a β₂-adrenoceptor agonist. The beneficial effect may be observed when the two active substances are administered simultaneously (either in a single pharmaceutical preparation or via separate preparations), or sequentially or separately via separate pharmaceutical preparations.

The pharmaceutical product of the present invention may, for example, be a pharmaceutical composition comprising the first and second active ingredients in admixture. Alternatively, the pharmaceutical product may, for example, be a kit comprising a preparation of the first active ingredient and a preparation of the second active ingredient and, optionally, instructions for the simultaneous, sequential or separate administration of the preparations to a patient in need thereof.

The first active ingredient in the combination of the present invention is a muscarinic antagonist selected from:
[2-((S)-Cyclohexyl-hydroxy-phenyl-methyl)-oxazol-5-ylmethyl]-dimethyl-(3-phenoxy-propyl)-ammonium salt,
[2-((R)-Cyclohexyl-hydroxy-phenyl-methyl)-oxazol-5-ylmethyl]-dimethyl-(3-phenoxy-propyl)-ammonium salt,
[2-((R)-Cyclohexyl-hydroxy-phenyl-methyl)-oxazol-5-ylmethyl]-dimethyl-(2-phenethyloxy-ethyl)-ammonium salt,
[2-((R)-Cyclohexyl-hydroxy-phenyl-methyl)-oxazol-5-ylmethyl]-[3-(3,4-dichloro-phenoxy)-propyl] dimethyl-ammonium salt,
[2-((R)-Cyclohexyl-hydroxy-phenyl-methyl)-oxazol-5-ylmethyl]-[2-(3,4-dichloro-benzyloxy)-ethyl]-dimethyl-ammonium salt; and
[2-(4-Chloro-benzyloxy)-ethyl]-[2-((R)-Cyclohexyl-hydroxy-phenyl-methyl)-oxazol-5-ylmethyl]-dimethyl-ammonium salt.

The muscarinic antagonists of the invention are selected members of a novel class of compound described in WO2007/017669 (PCT/GB2006/002956) which display high potency to the M3 receptor. The names of the muscarinic antagonists are IUPAC names generated by the Autonom 2000 plug in for IsisDraw Version 2.5, as supplied by MDL Information Systems Inc., based on the structures depicted in the examples, and stereochemistry assigned according to the Cahn-Ingold-Prelog system. For example, the name [2-((R)-Cyclohexyl-hydroxy-phenyl-methyl)-oxazol-5-ylmethyl]-dimethyl-(3-phenoxy-propyl)-ammonium, was generated from the structure:

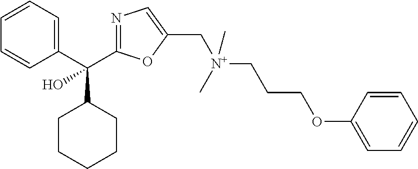

The muscarinic receptor antagonists of the present invention are ammonium salts. The salt anion may be any pharmaceutically acceptable anion of a mono or polyvalent (e.g. bivalent) acid. In an embodiment of the invention, the salt anion is selected from Chloride, bromide, iodide, sulfate, benzenesulfonate, toluenesulfonate (tosylate), napadisylate (naphthalene-1,5-disulfonate), edisylate (ethane-1,2-disulfonate), isethionate (2-hydroxyethylsulfonate), phosphate, acetate, citrate, lactate, tartrate, oleic, mesylate (methanesulfonate), maleate ((Z)-3-carboxy-acrylate), fumarate, succinate (3-carboxy-propionate), malate ((S)-3-carboxy-2-hydroxy-propionate), xinafoate and p-acetamidobenzoate.

In an embodiment of the invention, the muscarinic receptor antagonist is selected from
[2-((S)-Cyclohexyl-hydroxy-phenyl-methyl)-oxazol-5-ylmethyl]-dimethyl-(3-phenoxy-propyl)-ammonium bromide,
[2-((R)-Cyclohexyl-hydroxy-phenyl-methyl)-oxazol-5-ylmethyl]-dimethyl-(3-phenoxy-propyl)-ammonium bromide,
[2-((R)-Cyclohexyl-hydroxy-phenyl-methyl)-oxazol-5-ylmethyl]-dimethyl-(3-phenoxy-propyl)-ammonium tosylate,
[2-((R)-Cyclohexyl-hydroxy-phenyl-methyl)-oxazol-5-ylmethyl]-dimethyl-(3-phenoxy-propyl)-ammonium maleate,
[2-((R)-Cyclohexyl-hydroxy-phenyl-methyl)-oxazol-5-ylmethyl]-dimethyl-(3-phenoxy-propyl)-ammonium succinate,
[2-((R)-Cyclohexyl-hydroxy-phenyl-methyl)-oxazol-5-ylmethyl]-dimethyl-(3-phenoxy-propyl)-ammonium malate,
[2-((R)-Cyclohexyl-hydroxy-phenyl-methyl)-oxazol-5-ylmethyl]-dimethyl-(3-phenoxy-propyl)-ammonium napadisylate,
[2-((R)-Cyclohexyl-hydroxy-phenyl-methyl)-oxazol-5-ylmethyl]-dimethyl-(2-phenethyloxy-ethyl)-ammonium bromide,
[2-((R)-Cyclohexyl-hydroxy-phenyl-methyl)-oxazol-5-ylmethyl]-dimethyl-(2-phenethyloxy-ethyl)-ammonium napadisylate,
[2-(4-Chloro-benzyloxy)-ethyl]-[2-((R)-Cyclohexyl-hydroxy-phenyl-methyl)-oxazol-5-ylmethyl]-dimethyl-ammonium mesylate,
[2-((R)-Cyclohexyl-hydroxy-phenyl-methyl)-oxazol-5-ylmethyl]-[3-(3,4-dichloro-phenoxy)-propyl] dimethyl-ammonium bromide,
[2-((R)-Cyclohexyl-hydroxy-phenyl-methyl)-oxazol-5-ylmethyl]-[3-(3,4-dichloro-phenoxy)-propyl] dimethyl-ammonium napadisylate,
[2-((R)-Cyclohexyl-hydroxy-phenyl-methyl)-oxazol-5-ylmethyl]-[2-(3,4-dichloro-benzyloxy)-ethyl]-dimethyl-ammonium bromide,
[2-((R)-Cyclohexyl-hydroxy-phenyl-methyl)-oxazol-5-ylmethyl]-[2-(3,4-dichloro-benzyloxy)-ethyl]-dimethyl-ammonium napadisylate,
[2-(4-Chloro-benzyloxy)-ethyl]-[2-((R)-Cyclohexyl-hydroxy-phenyl-methyl)-oxazol-5-ylmethyl]-dimethyl-ammonium bromide,
[2-(4-Chloro-benzyloxy)-ethyl]-[2-((R)-Cyclohexyl-hydroxy-phenyl-methyl)-oxazol-5-ylmethyl]-dimethyl-ammonium napadisylate, and
[2-((R)-Cyclohexyl-hydroxy-phenyl-methyl)-oxazol-5-ylmethyl]-[2-(3,4-dichloro-benzyloxy)-ethyl] dimethyl-ammonium mesylate.

In an embodiment of the invention, the muscarinic receptor antagonist is in the form of a bromide or napadisylate salt.

In an embodiment of the invention, the muscarinic receptor antagonist is in the form of a napadisylate salt. When the muscarinic antagonist is a napadisylate salt the cation/anion ratio may vary, and for example may be 1:1 or 2:1 or a value between 1:1 and 2:1.

In an embodiment of the invention the muscarinic antagonist is in the form of a napadisylate salt wherein the napadisylate salt cation/anion ratio is 2:1. i.e. a hemi-napadisylate. Examples of muscarinic antagonists according to this embodiment include
[2-((R)-Cyclohexyl-hydroxy-phenyl-methyl)-oxazol-5-ylmethyl]-dimethyl-(3-phenoxy-propyl)-ammonium hemi-naphthalene-1,5-disulfonate,
[2-((R)-Cyclohexyl-hydroxy-phenyl-methyl)-oxazol-5-ylmethyl]-dimethyl-(2-phenethyloxy-ethyl)-ammonium hemi-naphthalene-1,5-disulfonate,
[2-((R)-Cyclohexyl-hydroxy-phenyl-methyl)-oxazol-5-ylmethyl]-[3-(3,4-dichloro-phenoxy)-propyl] dimethyl-ammonium hemi-naphthalene-1,5-disulfonate,
[2-((R)-Cyclohexyl-hydroxy-phenyl-methyl)-oxazol-5-ylmethyl]-[2-(3,4-dichloro-benzyloxy)-ethyl]-dimethyl-ammonium hemi-naphthalene-1,5-disulfonate, and
[2-(4-Chloro-benzyloxy)-ethyl]-[2-((R)-Cyclohexyl-hydroxy-phenyl-methyl)-oxazol-5-ylmethyl]-dimethyl-ammonium hemi-naphthalene-1,5-disulfonate.

In an embodiment of the invention, the muscarinic receptor antagonist is in the form of a bromide salt.

The second active ingredient in the combination of the present invention is a β₂-adrenoceptor agonist. The β₂-adrenoceptor agonist of the present invention may be any compound or substance capable of stimulating the β₂-receptors and acting as a bronchodilator. In the context of the present specification, unless otherwise stated, any reference to a β₂-adrenoceptor agonist includes active salts, solvates or derivatives that may be formed from said β₂-adrenoceptor agonist and any enantiomers and mixtures thereof. Examples of possible salts or derivatives of β₂-adrenoceptor agonist are acid addition salts such as the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, maleic acid, and pharmaceutically acceptable esters (e.g. C₁-C₆ alkyl esters). The β₂-agonists may also be in the form of solvates, e.g. hydrates.

Examples of a β₂-adrenoceptor agonist that may be used in the pharmaceutical product according to this embodiment include metaproterenol, isoproterenol, isoprenaline, albuterol, salbutamol (e.g. as sulphate), formoterol (e.g. as fumarate), salmeterol (e.g. as xinafoate), terbutaline, orciprenaline, bitolterol (e.g. as mesylate), pirbuterol or indacaterol. The β₂-adrenoceptor agonist of this embodiment may be a long-acting β₂-agonist (i.e. a β₂-agonist with activity that persists for more than 24 hours), for example salmeterol (e.g. as xinafoate), formoterol (e.g. as fumarate), bambuterol (e.g. as hydrochloride), carmoterol (TA 2005, chemically identified as 2(1H)-Quinolone, 8-hydroxy-5-[1-hydroxy-2-[[2-(4-methoxy-phenyl)-1-methylethyl]-amino]ethyl]-monohydrochloride, [R-(R*,R*)] also identified by Chemical Abstract Service Registry Number 137888-11-0 and disclosed in U.S. Pat. No. 4,579,854), indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555, indole derivatives as disclosed in WO 2004/032921, in US 2005/222144, compounds GSK 159797, GSK 159802, GSK 597901, GSK 642444 and GSK 678007.

In an embodiment of the present invention, the β₂-adrenoceptor agonist is formoterol. The chemical name for formoterol is N-[2-hydroxy-5-[(1)-1-hydroxy-2-[[(1)-2-(4-methoxyphenyl)-1-methylethyl]amino]ethyl]phenyl]-formamide. The preparation of formoterol is described, for example, in WO 92/05147. In one aspect of this embodiment, the β₂-adrenoceptor agonist is formoterol fumarate. It will be understood that the invention encompasses the use of all optical isomers of formoterol and mixtures thereof including racemates. Thus for example, the term formoterol encompasses N-[2-hydroxy-5-[(1R)-1-hydroxy-2-[[(1R)-2-(4-methoxyphenyl)-1-methylethyl]amino]ethyl]phenyl]-formamide, N-[2-hydroxy-5-[(1S)-1-hydroxy-2-[[(1S)-2-(4-methoxyphenyl)-1-methylethyl]amino]ethyl]phenyl]-formamide and a mixture of such enantiomers, including a racemate.

In an embodiment of the invention, the β₂-adrenoceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide, and
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
   or a pharmaceutically acceptable salt thereof. The β₂-adrenoceptor agonists according to this embodiment may be prepared as described in the experimental preparation section of the present application. The names of the β₂-adrenoceptor agonists of this embodiment are IUPAC names generated by the IUPAC NAME, ACD Labs Version 8 naming package.

In a further embodiment of the invention, the β₂-adrenoceptor agonist is selected from:
N-[2-(Diethylamino)ethyl]-N-(2-[2-(4-hydroxy-2-oxo-2,3-dihydro-1,3-benzothiazol-7-yl)ethyl]amino)ethyl)-3-[2-(1-naphthyl)ethoxy]propanamide dihydrobromide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide, and
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide.

In an embodiment of the invention, the muscarinic receptor antagonist is a [2-((R)-cyclohexyl-hydroxy-phenyl-methyl)-oxazol-5-ylmethyl]-dimethyl-(3-phenoxy-propyl)-ammonium salt, and the β₂-adrenoceptor agonist is formoterol (e.g. as fumarate). In one aspect of this embodiment, the muscarinic receptor antagonist is [2-((R)-cyclohexyl-hydroxy-phenyl-methyl)-oxazol-5-ylmethyl]-dimethyl-(3-phenoxy-propyl)-ammonium bromide. In another aspect of this embodiment, the muscarinic receptor antagonist is [2-((R)-cyclohexyl-hydroxy-phenyl-methyl)-oxazol-5-ylmethyl]-dimethyl-(3-phenoxy-propyl)-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-dimethyl-(2-phenethyloxy-ethyl)-ammonium salt, and the β₂-adrenoceptor agonist is formoterol (e.g. as fumarate). In one aspect of this embodiment, the muscarinic receptor antagonist is [2-((R)-cyclohexyl-hydroxy-phenyl-methyl)-oxazol-5-ylmethyl]-dimethyl-(2-phenethyloxy-ethyl)-ammonium bromide. In another aspect of this embodiment, the muscarinic receptor antagonist is [2-((R)-cyclohexyl-hydroxy-phenyl-methyl)-oxazol-5-ylmethyl]-dimethyl-(2-phenethyloxy-ethyl)-ammonium napadisylate (e.g. hemi-naphthalene-1,5-disulfonate).

In an embodiment of the invention, the muscarinic receptor antagonist is a [[2-((R)-cyclohexyl-hydroxy-phenyl-methyl)-oxazol-5-ylmethyl]-[3-(3,4-dichloro-phenoxy)-propyl] dimethyl-ammonium salt and the β₂-adrenoceptor agonist is formoterol (e.g. as fumarate). In one aspect of this embodiment, the muscarinic receptor antagonist is [[2-((R)-cyclohexyl-hydroxy-phenyl-methyl)-oxazol-5-ylmethyl]-[3-(3,4-dichloro-phenoxy)-propyl] dimethyl-ammonium bromide. In another aspect of this embodiment, the muscarinic receptor antagonist is [2-((R)-cyclohexyl-hydroxy-phenyl-methyl)-oxazol-5-ylmethyl]-[3-(3,4-dichloro-phenoxy)-propyl] dimethyl-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-[2-(3,4-dichloro-benzyloxy)-ethyl]-dimethyl-ammonium salt and the β₂-adrenoceptor agonist is formoterol (e.g. as fumarate). In one aspect of this embodiment, the muscarinic receptor antagonist is [2-((R)-cyclohexyl-hydroxy-phenyl-methyl)-oxazol-5-ylmethyl]-[2-(3,4-dichloro-benzyloxy)-ethyl]-dimethyl-ammonium bromide. In another aspect of this embodiment, the muscarinic receptor antagonist is [2-((R)-cyclohexyl-hydroxy-phenyl-methyl)-oxazol-5-ylmethyl]-[2-(3,4-dichloro-benzyloxy)-ethyl]-dimethyl-ammonium napadisylate (e.g. hemi-naphthalene-1,5-disulfonate).

In an embodiment of the invention, the muscarinic receptor antagonist is a [2-(4-chloro-benzyloxy)-ethyl]-[2-((R)-cyclohexyl-hydroxy-phenyl-methyl)-oxazol-5-ylmethyl]-dimethyl-ammonium salt and the β₂-adrenoceptor agonist is formoterol (e.g. as fumarate). In one aspect of this embodiment, the muscarinic receptor antagonist is [2-(4-chloro-benzyloxy)-ethyl]-[2-((R)-cyclohexyl-hydroxy-phenyl-methyl)-oxazol-5-ylmethyl]-dimethyl-ammonium bromide. In another aspect of this embodiment, the muscarinic receptor antagonist is [2-(4-chloro-benzyloxy)-ethyl]-[2-((R)-cyclohexyl-hydroxy-phenyl-methyl)-oxazol-5-ylmethyl]-dimethyl-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-dimethyl-(3-phenoxy-propyl)-ammonium salt, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-dimethyl-(3-phenoxy-propyl)-ammonium bromide. In another aspect of this embodiment, the muscarinic receptor antagonist is [2-((R)-cyclohexyl-hydroxy-phenyl-methyl)-oxazol-5-ylmethyl]-dimethyl-(3-phenoxy-propyl)-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-dimethyl-(2-phenethyloxy-ethyl)-ammonium salt, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-dimethyl-(2-phenethyloxy-ethyl)-ammonium bromide. In another aspect of this embodiment, the muscarinic receptor antagonist is [2-((R)-cyclohexyl-hydroxy-phenyl-methyl)-oxazol-5-ylmethyl]-dimethyl-(2-phenethyloxy-ethyl)-ammonium napadisylate (e.g. hemi-naphthalene-1,5-disulfonate).

In an embodiment of the invention, the muscarinic receptor antagonist is a [[2-((R)-cyclohexyl-hydroxy-phenyl-methyl)-oxazol-5-ylmethyl]-[3-(3,4-dichloro-phenoxy)-propyl] dimethyl-ammonium salt and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is [[2-((R)-cyclohexyl-hydroxy-phenyl-methyl)-oxazol-5-ylmethyl]-[3-(3,4-dichloro-phenoxy)-propyl] dimethyl-ammonium bromide. In another aspect of this embodiment, the muscarinic receptor antagonist is [2-((R)-cyclohexyl-hydroxy-phenyl-methyl)-oxazol-5-ylmethyl]-[3-(3,4-dichloro-phenoxy)-propyl] dimethyl-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-[2-(3,4-dichloro-benzyloxy)-ethyl]-dimethyl-ammonium salt and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-[2-(3,4-dichloro-benzyloxy)-ethyl]-dimethyl-ammonium bromide. In another aspect of this embodiment, the muscarinic receptor antagonist is [2-((R)-cyclohexyl-hydroxy-phenyl-methyl)-oxazol-5-ylmethyl]-[2-(3,4-dichloro-benzyloxy)-ethyl]-dimethyl-ammonium napadisylate (e.g. hemi-naphthalene-1,5-disulfonate).

In an embodiment of the invention, the muscarinic receptor antagonist is a [2-(4-chloro-benzyloxy)-ethyl]-[2-((R)-cyclohexyl-hydroxy-phenyl-methyl)-oxazol-5-ylmethyl]-dimethyl-ammonium salt and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is [2-(4-chloro-benzyloxy)-ethyl]-[2-((R)-cyclohexyl-hydroxy-phenyl-methyl)-oxazol-5-ylmethyl]-dimethyl-ammonium bromide. In another aspect of this embodiment, the muscarinic receptor antagonist is [2-(4-chloro-benzyloxy)-ethyl]-[2-((R)-cyclohexyl-hydroxy-phenyl-methyl)-oxazol-5-ylmethyl]-dimethyl-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-dimethyl-(3-phenoxy-propyl)-ammonium salt, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-dimethyl-(3-phenoxy-propyl)-ammonium bromide. In another aspect of this embodiment, the muscarinic receptor antagonist is [2-((R)-cyclohexyl-hydroxy-phenyl-methyl)-oxazol-5-ylmethyl]-dimethyl-(3-phenoxy-propyl)-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-dimethyl-(2-phenethyloxy-ethyl)-ammonium salt, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-dimethyl-(2-phenethyloxy-ethyl)-ammonium bromide. In another aspect of this embodiment, the muscarinic receptor antagonist is [2-((R)-cyclohexyl-hydroxy-phenyl-methyl)-oxazol-5-ylmethyl]-dimethyl-(2-phenethyloxy-ethyl)-ammonium napadisylate (e.g. hemi-naphthalene-1,5-disulfonate).

In an embodiment of the invention, the muscarinic receptor antagonist is a [[2-((R)-cyclohexyl-hydroxy-phenyl-methyl)-oxazol-5-ylmethyl]-[3-(3,4-dichloro-phenoxy)-propyl] dimethyl-ammonium salt and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a pharmaceutically acceptable salt thereof (e.g. dihydrobromide). In one aspect of this embodiment, the muscarinic receptor antagonist is [[2-((R)-cyclohexyl-hydroxy-phenyl-methyl)-oxazol-5-ylmethyl]-[3-(3,4-dichloro-phenoxy)-propyl] dimethyl-ammonium bromide. In another aspect of this embodiment, the muscarinic receptor antagonist is [2-((R)-cyclohexyl-hydroxy-phenyl-methyl)-oxazol-5-ylmethyl]-[3-(3,4-dichloro-phenoxy)-propyl] dimethyl-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-[2-(3,4-dichloro-benzyloxy)-ethyl]-dimethyl-ammonium salt and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-[2-(3,4-dichloro-benzyloxy)-ethyl]-dimethyl-ammonium bromide. In another aspect of this embodiment, the muscarinic receptor antagonist is [2-((R)-cyclohexyl-hydroxy-phenyl-methyl)-oxazol-5-ylmethyl]-[2-(3,4-dichloro-benzyloxy)-ethyl]-dimethyl-ammonium napadisylate (e.g. hemi-naphthalene-1,5-disulfonate).

In an embodiment of the invention, the muscarinic receptor antagonist is a [2-(4-chloro-benzyloxy)-ethyl]-[2-((R)-cyclohexyl-hydroxy-phenyl-methyl)-oxazol-5-ylmethyl]-dimethyl-ammonium salt and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide or a pharmaceutically acceptable salt thereof (e.g. dihydrobromide). In one aspect of this embodiment, the muscarinic receptor antagonist is [2-(4-chloro-benzyloxy)-ethyl]-[2-((R)-cyclohexyl-hydroxy-phenyl-methyl)-oxazol-5-ylmethyl]-dimethyl-ammonium bromide. In another aspect of this embodiment, the muscarinic receptor antagonist is [2-(4-chloro-benzyloxy)-ethyl]-[2-((R)-cyclohexyl-hydroxy-phenyl-methyl)-oxazol-5-ylmethyl]-dimethyl-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-dimethyl-(3-phenoxy-propyl)-ammonium salt, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-dimethyl-(3-phenoxy-propyl)-ammonium bromide. In another aspect of this embodiment, the muscarinic receptor antagonist is [2-((R)-cyclohexyl-hydroxy-phenyl-methyl)-oxazol-5-ylmethyl]-dimethyl-(3-phenoxy-propyl)-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-dimethyl-(2-phenethyloxy-ethyl)-ammonium salt, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-dimethyl-(2-phenethyloxy-ethyl)-ammonium bromide. In another aspect of this embodiment, the muscarinic receptor antagonist is [2-((R)-cyclohexyl-hydroxy-phenyl-methyl)-oxazol-5-ylmethyl]-dimethyl-(2-phenethyloxy-ethyl)-ammonium napadisylate (e.g. hemi-naphthalene-1,5-disulfonate).

In an embodiment of the invention, the muscarinic receptor antagonist is a [(2-((R)-cyclohexyl-hydroxy-phenyl-methyl)-oxazol-5-ylmethyl]-[3-(3,4-dichloro-phenoxy)-propyl] dimethyl-ammonium salt and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is [[2-((R)-cyclohexyl-hydroxy-phenyl-methyl)-oxazol-5-ylmethyl]-[3-(3,4-dichloro-phenoxy)-propyl] dimethyl-ammonium bromide. In another aspect of this embodiment, the muscarinic receptor antagonist is [2-((R)-cyclohexyl-hydroxy-phenyl-methyl)-oxazol-5-ylmethyl]-[3-(3,4-dichloro-phenoxy)-propyl] dimethyl-ammonium napadisylate (e.g. hemi-naphthalene-1,5-disulfonate).

In an embodiment of the invention, the muscarinic receptor antagonist is a [2-((R)-cyclohexyl-hydroxy-phenyl-methyl)-oxazol-5-ylmethyl]-[2-(3,4-dichloro-benzyloxy)-ethyl]-dimethyl-ammonium salt and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is [2-((R)-cyclohexyl-hydroxy-phenyl-methyl)-oxazol-5-ylmethyl]-[2-(3,4-dichloro-benzyloxy)-ethyl]-dimethyl-ammonium bromide. In another aspect of this embodiment, the muscarinic receptor antagonist is [2-((R)-cyclohexyl-hydroxy-phenyl-methyl)-oxazol-5-ylmethyl]-[2-(3,4-dichloro-benzyloxy)-ethyl]-dimethyl-ammonium napadisylate (e.g. hemi-naphthalene-1,5-disulfonate).

In an embodiment of the invention, the muscarinic receptor antagonist is a [2-(4-chloro-benzyloxy)-ethyl]-[2-((R)-cyclohexyl-hydroxy-phenyl-methyl)-oxazol-5-ylmethyl]-dimethyl-ammonium salt and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is [2-(4-chloro-benzyloxy)-ethyl]-[2-((R)-cyclohexyl-hydroxy-phenyl-methyl)-oxazol-5-ylmethyl]-dimethyl-ammonium bromide. In another aspect of this embodiment, the muscarinic receptor antagonist is [2-(4-chloro-benzyloxy)-ethyl]-[2-((R)-cyclohexyl-hydroxy-phenyl-methyl)-oxazol-5-ylmethyl]-dimethyl-ammonium napadisylate (e.g. hemi-naphthalene-1,5-disulfonate).

The combination of the present invention may provide a beneficial therapeutic effect in the treatment of respiratory diseases. Examples of such possible effects include improvements in one or more of the following parameters: reducing inflammatory cell influx into the lung, mild and severe exacerbations, FEV₁ (forced expiratory volume in one second), vital capacity (VC), peak expiratory flow (PEF), symptom scores and Quality of Life.

The muscarinic antagonist (first active ingredient) and β₂-adrenoceptor agonist (second active ingredient) of the present invention may be administered simultaneously, sequentially or separately to treat respiratory diseases. By sequential it is meant that the active ingredients are administered, in any order, one immediately after the other. They may still have the desired effect if they are administered separately, but when administered in this manner they will generally be administered less than 4 hours apart, more conveniently less than two hours apart, more conveniently less than 30 minutes apart and most conveniently less than 10 minutes apart.

The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may also be administered topically (to the lung and/or airways) in the form of solutions, suspensions, aerosols and dry powder formulations. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants. As will be understood by those skilled in the art, the most appropriate method of administering the active ingredients is dependent on a number of factors.

In one embodiment of the present invention the active ingredients are administered via separate pharmaceutical preparations. Therefore, in one aspect, the present invention provides a kit comprising a preparation of a first active ingredient which is a muscarinic antagonist according to the present invention, and a preparation of a second active ingredient which is a β₂-adrenoceptor agonist, and optionally instructions for the simultaneous, sequential or separate administration of the preparations to a patient in need thereof.

In another embodiment the active ingredients may be administered via a single pharmaceutical composition. Therefore, the present invention further provides a pharmaceutical composition comprising, in admixture, a first active ingredient, which is a muscarinic antagonist according to the present invention, and a second active ingredient, which is a β₂-adrenoceptor agonist.

The pharmaceutical compositions of the present invention may be prepared by mixing the muscarinic antagonist (first active ingredient) with a β₂-adrenoceptor agonist (second active ingredient) and a pharmaceutically acceptable adjuvant, diluent or carrier. Therefore, in a further aspect of the present invention there is provided a process for the preparation of a pharmaceutical composition, which comprises mixing a muscarinic antagonist according to the present invention with a β₂-adrenoceptor agonist and a pharmaceutically acceptable adjuvant, diluent or carrier.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, muscarinic antagonist according to the present invention is administered via inhalation. When administered via inhalation the dose of the muscarinic antagonist according to the present invention will generally be in the range of from 0.1 microgram (μg) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In one embodiment of the present invention the β₂-adrenoceptor agonist may conveniently be administered by inhalation. When administered via inhalation the dose of the β₂-agonist will generally be in the range of from 0.1 to 50 μg, 0.1 to 40 μg, 0.1 to 30 μg, 0.1 to 20 μg, 0.1 to 10 μg, 5 to 10 μg, 5 to 50 μg, 5 to 40 μg, 5 to 30 μg, 5 to 20 μg, 5 to 10 μg, 10 to 50 μg, 10 to 40 μg 10 to 30 μg, or 10 to 20 μg. The dose will generally be administered from 1 to 4 times a day, conveniently once or twice a day, and most conveniently once a day.

In one embodiment, the present invention provides a pharmaceutical product comprising, in combination, a first active ingredient which is a muscarinic antagonist according to the present invention, and a second active ingredient which is a β₂-adrenoceptor agonist, wherein each active ingredient is formulated for inhaled administration.

The active ingredients of the present invention are conveniently administered via inhalation (e.g. topically to the lung and/or airways) in the form of solutions, suspensions, aerosols and dry powder formulations. For example metered dose inhaler devices may be used to administer the active ingredients, dispersed in a suitable propellant and with or without additional excipients such as ethanol, surfactants, lubricants or stabilising agents. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose formulations.

Dry powder formulations and pressurized HFA aerosols of the active ingredients may be administered by oral or nasal inhalation. For inhalation, the compound is desirably finely divided. The finely divided compound preferably has a mass median diameter of less than and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

The combination of the present invention is useful in the treatment or prevention of respiratory-tract disorders such as chronic obstructive pulmonary disease (COPD), chronic bronchitis of all types (including dyspnoea associated therewith), asthma (allergic and non-allergic; ‘wheezy-infant syndrome’), adult/acute respiratory distress syndrome (ARDS), chronic respiratory obstruction, bronchial hyperactivity, pulmonary fibrosis, pulmonary emphysema, and allergic rhinitis, exacerbation of airway hyperreactivity consequent to other drug therapy, particularly other inhaled drug therapy or pneumoconiosis (for example aluminosis, anthracosis, asbestosis, chalicosis, ptilosis, siderosis, silicosis, tabacosis and byssinosis).

Dry powder inhalers may be used to administer the active ingredients, alone or in combination with a pharmaceutically acceptable carrier, in the later case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices are available.

The present invention further provides a pharmaceutical product, kit or pharmaceutical composition according to the invention for simultaneous, sequential or separate use in therapy.

The present invention further provides the use of a pharmaceutical product, kit or pharmaceutical composition according to the invention in the treatment of a respiratory disease, in particular chronic obstructive pulmonary disease or asthma.

The present invention further provides the use of a pharmaceutical product, kit or pharmaceutical composition according to the invention in the manufacture of a medicament for the treatment of a respiratory disease, in particular chronic obstructive pulmonary disease or asthma.

The present invention still further provides a method of treating a respiratory disease which comprises simultaneously, sequentially or separately administering:

(a) a (therapeutically effective) dose of a first active ingredient which is a muscarinic antagonist according to the present invention; and

(b) a (therapeutically effective) dose of a second active ingredient which is a β₂-adrenoceptor agonist;

to a patient in need thereof.

In the context of the present specification, the term “therapy” also includes “prophylaxis” unless there are specific indications to the contrary. The terms “therapeutic” and “therapeutically” should be construed accordingly. Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

The pharmaceutical product, kit or composition of the present invention may optionally comprise a third active ingredient which third active ingredient is a substance suitable for use in the treatment of respiratory diseases. Examples of a third active ingredient that may be incorporated into the present invention include
a phosphodiesterase inhibitor,
a modulator of chemokine receptor function,
an inhibitor of kinase function,
a protease inhibitor,
a steroidal glucocorticoid receptor agonist, and a
a non-steroidal glucocorticoid receptor agonist.

Examples of a phosphodiesterase inhibitor that may be used as a third active ingredient according to this embodiment include a PDE4 inhibitor such as an inhibitor of the isoform PDE4D, a PDE3 inhibitor and a PDE5 inhibitor. Examples include the compounds
(Z)-3-(3,5-dichloro-4-pyridyl)-2-[4-(2-indanyloxy-5-methoxy-2-pyridyl]propenenitrile,
N-[9-amino-4-oxo-1-phenyl-3,4,6,7-tetrahydropyrrolo[3,2,1-jk][1,4]benzodiazepin-3(R)-yl]pyridine-3-carboxamide (CI-1044)
3-(benzyloxy)-1-(4-fluorobenzyl)-N-[3-(methylsulphonyl)phenyl]-1H-indole-2-carboxamide,
(1S-exo)-5-[3-(bicyclo[2.2.1]hept-2-yloxy)-4-methoxyphenyl]tetrahydro-2(1H)-pyrimidinone (Atizoram),
N-(3,5,dichloro-4-pyridinyl)-2-[1-(4-fluorobenzyl)-5-hydroxy-1H-indol-3-yl]-2-oxoacetamide (AWD-12-281),
β-[3-(cyclopentyloxy)-4-methoxyphenyl]-1,3-dihydro-1,3-dioxo-2H-isoindole-2-propanamide (CDC-801),
N-[9-methyl-4-oxo-1-phenyl-3,4,6,7-tetrahydropyrrolo[3,2,1-jk][1,4]benzodiazepin-3(R)-yl]pyridine-4-carboxamide (CI-1018),
cis-[4-cyano-4-(3-cyclopentyloxy-4-methoxyphenyl)cyclohexane-1-carboxylic acid (Cilomilast)
8-amino-1,3-bis(cyclopropylmethyl)xanthine (Cipamfylline)
N-(2,5-dichloro-3-pyridinyl)-8-methoxy-5-quinolinecarboxamide (D-4418),
5-(3,5-di-tert-butyl-4-hydroxybenzylidene)-2-iminothiazolidin-4-one (Darbufelone),
2-methyl-1-[2-(1-methylethyl)pyrazolo[1,5-a]pyridin-3-yl]-1-propanone (Ibudilast),
2-(2,4-dichlorophenylcarbonyl)-3-ureidobenzofuran-6-yl methanesulphonate (Lirimilast),
(−)-(R)-5-(4-methoxy-3-propoxyphenyl)-5-methyloxazolidin-2-one (Mesopram),
(−)-cis-9-ethoxy-8-methoxy-2-methyl-1,2,3,4,4a,10b-hexahydro-6-(4-diisopropylaminocarbonylphenyl)-benzo[c][1,6]naphthyridine (Pumafentrine),
3-(cyclopropylmethoxy)-N-(3,5-dichloro-4-pyridyl)-4-(difluoromethoxy)benzamide (Roflumilast),
the N-oxide of Roflumilast,
5,6-diethoxybenzo[b]thiophene-2-carboxylic acid (Tibenelast)
2,3,6,7-tetrahydro-2-(mesitylimino)-9,10-dimethoxy-3-methyl-4H-pyrimido[6,1-a]isoquinolin-4-one (trequinsin) and
3-[[3-(cyclopentyloxy)-4-methoxyphenyl]-methyl]-N-ethyl-8-(1-methylethyl)-3H-purine-6-amine (V-11294A).

Examples of a modulator of chemokine receptor function that may be used as a third active ingredient according to this embodiment include a CCR3 receptor antagonist, a CCR4 receptor antagonist, a CCR5 receptor antagonist and a CCR8 receptor antagonist.

Examples of an inhibitor of kinase function that may be used as a third active ingredient according to this embodiment include a p38 kinase inhibitor and an IKK inhibitor.

Examples of a protease inhibitor that may be used as a third active ingredient according to this embodiment include an inhibitor of neutrophil elastase or an inhibitor of MMP12.

Examples of a steroidal glucocorticoid receptor agonist that may be used as a third active ingredient according to this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

Examples of a modulator of a non-steroidal glucocorticoid receptor agonist that may be used as a third active ingredient according to this embodiment include those described in WO2006/046916.

The invention is illustrated by the following non-limiting Examples. In the Examples the following Figures are presented:

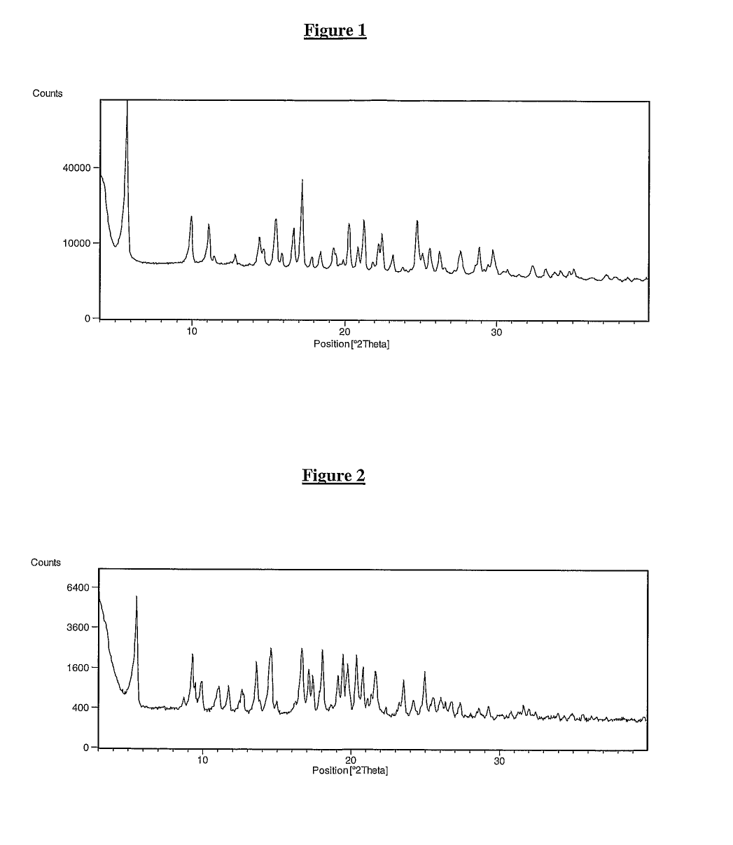

FIG. 1: X-ray powder diffraction pattern of Muscarinic Antagonist 2 (MA2) [2-((R)-Cyclohexyl-hydroxy-phenyl-methyl)-oxazol-5-ylmethyl]-dimethyl-(3-phenoxy-propyl)-ammonium bromide: Crystalline Form A

FIG. 2: X-ray powder diffraction pattern of Muscarinic Antagonist 7 (MA7) [2-((R)-Cyclohexyl-hydroxy-phenyl-methyl)-oxazol-5-ylmethyl]-dimethyl-(3-phenoxy-propyl)-ammonium hemi-naphthalene-1,5-disulfonate: Crystalline Form 1

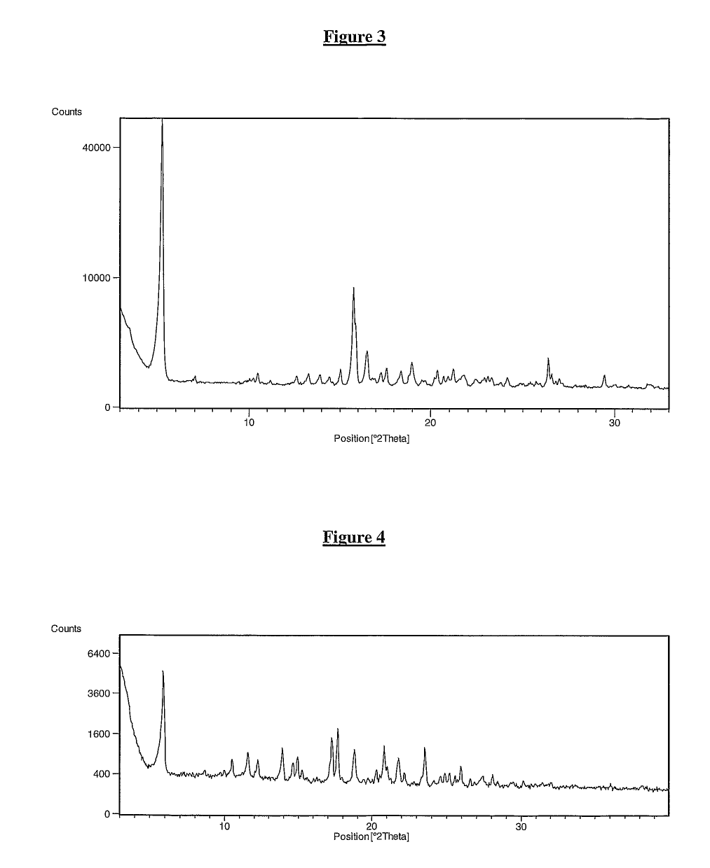

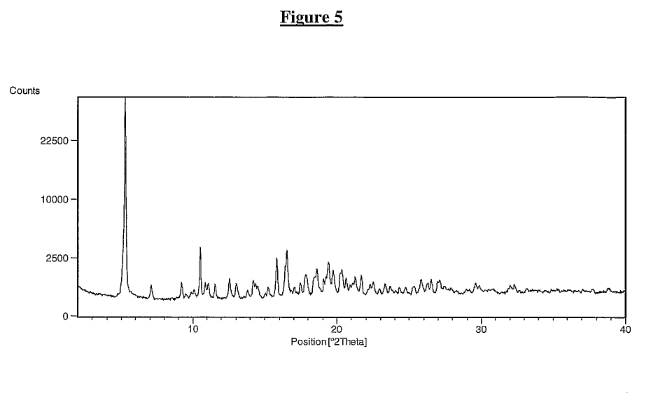

FIG. 3: X-ray powder diffraction pattern of Muscarinic Antagonist 7 (MA7): Crystalline Form 2

FIG. 4: X-ray powder diffraction pattern of Muscarinic Antagonist 7 (MA7): Crystalline Form 3

FIG. 5: X-ray powder diffraction pattern of Muscarinic Antagonist 11 (MA11) [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate: Crystalline Form A

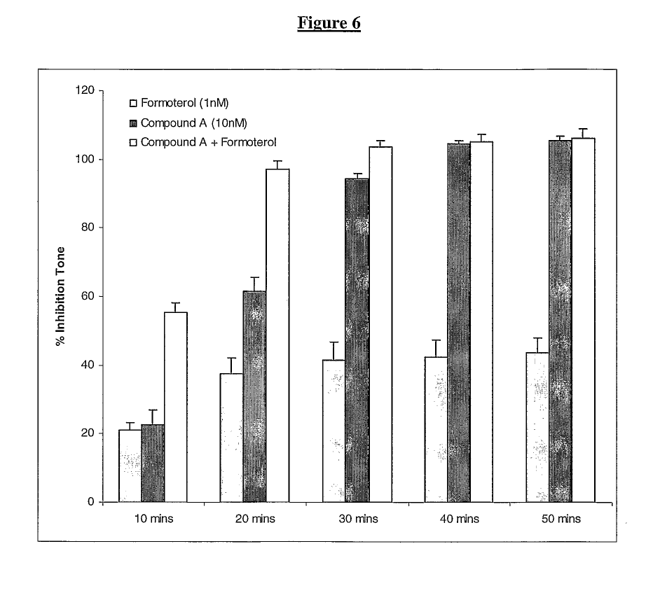

FIG. 6: % Inhibition of 1 μM Methacholine induced tone by Formoterol (1 nM), Compound A (MA2) (10 nM) and Compound A (10 nM) in the presence of Formoterol (1 nM) in guinea pig trachea in vitro

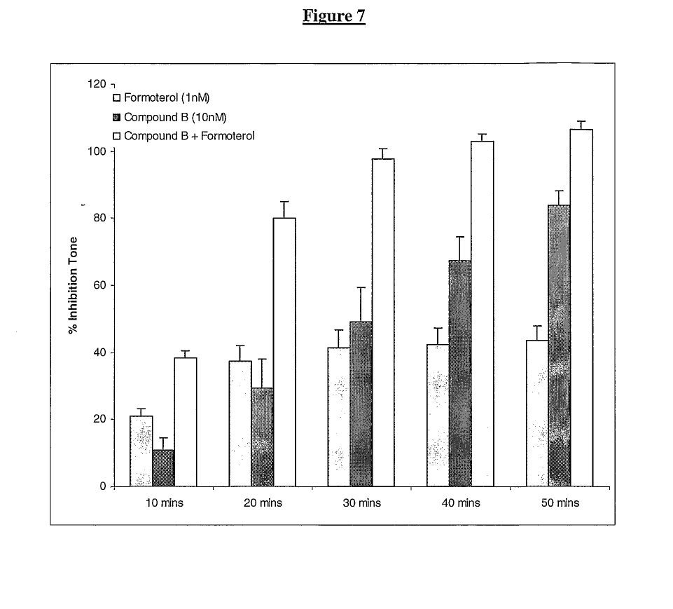

FIG. 7: % Inhibition of 1 μM Methacholine induced tone by Formoterol (1 nM), Compound B (MA11) (10 nM) and Compound B (10 nM) in the presence of Formoterol (1 nM) in guinea pig trachea in vitro

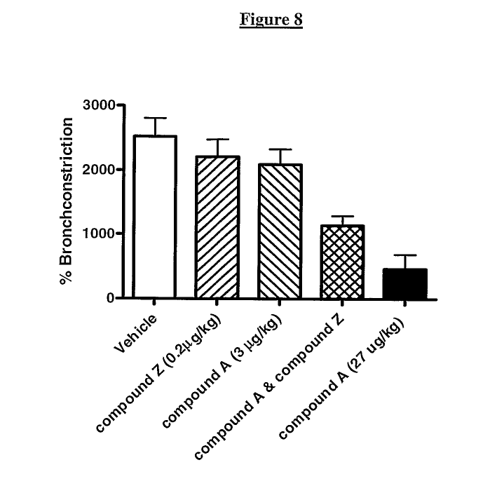

FIG. 8: Methacholine-induced bronchoconstriction in the guinea pig: 3 μg/kg and 27 μg/kg Compound A (BA1), 0.2 μg/kg Compound Z (MA2) or a combination of 3 μg/kg Compound A and 0.2 μg/kg Compound Z.

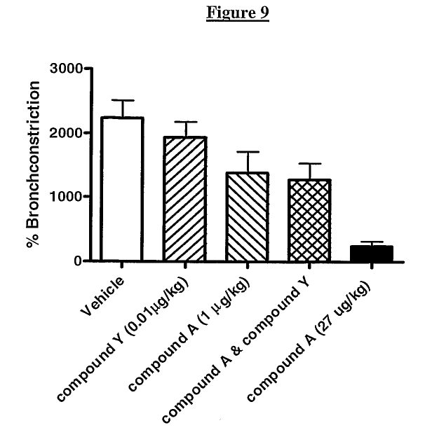

FIG. 9: Methacholine-induced bronchoconstriction in the guinea pig: 1 μg/kg and 27 μg/kg Compound A (BA1), 0.01 μg/kg Compound Y (MA11) or a combination of 1 μg/kg Compound A and 0.01 μg/kg Compound Y

### PREPARATION OF MUSCARINIC ANTAGONISTS

Muscarinic antagonists according to the present invention may be prepared as follows. Alternative salts to those described herein may be prepared by conventional chemistry using methods analogous to those described.

General Experimental Details for Preparation of Muscarinic Antagonists

Unless otherwise stsated the following general conditions were used in the preparation of the Muscarinic Antagonists

All reactions were carried out under an atmosphere of nitrogen unless specified otherwise.

NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz. Shifts are given in ppm relative to tetramethylsilane.

Where products were purified by column chromatography, ‘flash silica’ refers to silica gel for chromatography, 0.035 to 0.070 mm (220 to 440 mesh) (e.g. Fluka silica gel 60), and an applied pressure of nitrogen up to 10 p.s.i accelerated column elution. Where thin layer chromatography (TLC) has been used, it refers to silica gel TLC using plates, typically 3×6 cm silica gel on aluminium foil plates with a fluorescent indicator (254 nm), (e.g. Fluka 60778). All solvents and commercial reagents were used as received.

All compounds containing a basic centre(s), which were purified by HPLC, were obtained as the TFA salt unless otherwise stated.

Preparative HPLC Conditions:

C18-reverse-phase column (100×22.5 mm i.d. Genesis column with 7 μm particle size). to UV detection at 230 nm.

LC/MS Systems

The Liquid Chromatography Mass Spectroscopy (LC/MS) systems used:

LC-MS Method 1

Waters Platform LCT with a C18-reverse-phase column (100×3.0 mm Higgins Clipeus with 5 μm particle size), elution with A: water+0.1% formic acid; B: acetonitrile+0.1% formic acid. Gradient:

| Gradient - Time | flow mL/min | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Detection—MS, ELS, UV (100 μl split to MS with in-line UV detector at 254 nm)

MS ionisation method—Electrospray (positive ion)

LC-MS Method 2

Waters Platform LC with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow mL/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection—MS, ELS, UV (100 μl split to MS with in-line UV detector)

MS ionisation method—Electrospray (positive and negative ion)

LC-MS Method 3

Waters Micromass ZQ with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow mL/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection—MS, ELS, UV (100 μl split to MS with in-line UV detector)

MS ionisation method—Electrospray (positive and negative ion)

LC-MS Method 4

Waters Micromass ZQ with a C18-reverse-phase column (100×3.0 mm Higgins Clipeus with 5 μm particle size), elution with A: water+0.1% formic acid; B: acetonitrile+0.1% formic acid. Gradient:

| Gradient - Time | flow mL/min | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Detection—MS, ELS, UV (100 p. 1 split to MS with in-line UV detector at 254 nm)

MS ionisation method—Electrospray (positive ion)

X-Ray Powder Diffraction (XRPD) patterns were collected, on a high resolution Philips X-Pert MPD machine in reflection mode and θ-2θ configuration, over the scan range 2° to 40° 2θ with 100-second exposure per 0.03° increment. The X-rays were generated by a copper tube operated at 45 kV and 40 mA. The wavelengths of the direct beam X-rays was 1.5406 Å (K_{α1}) as a monochromator was used. The data was collected on zero background holders on which ˜2 mg of the compound was placed. The holder (provided by PANalytical) was made from a single crystal of silicon, which had been cut along a non-diffracting plane in the 2° to 40° 2θ range and then polished on an optically flat finish. The X-rays incident upon this surface were negated by Bragg extinction. Raw data were stored electronically and evaluation was performed on raw or smoothed diffraction patterns. XRPD were recorded at ambient temperature and relative humidity.

Differential scanning calorimetry (DSC) thermograms were measured using a TA Q1000 Differential Scanning calorimeter, with aluminium pans and pierced lids. The sample weights varied between 0.5 to 5 mg. The procedure was carried out under a flow of nitrogen gas (50 ml/min) and the temperature studied from 25 to 300° C. at a constant rate of temperature increase of 10° C. per minute.

Thermogravimetric analysis (TGA) thermograms were measured using a TA Q500 Thermogravimetric Analyser, with platinum pans. The sample weights varied between 1 and 5 mg. The procedure was carried out under a flow of nitrogen gas (60 ml/min) and the temperature studied from 25 to 200° C. at a constant rate of temperature increase of 10° C. per minute.

GVS profiles were measured using a Dynamic Vapour Sorption DVS-1 instrument. The solid sample ca. 1-5 mg was placed into a glass vessel and the weight of the sample was recorded during a dual cycle step method (40 to 90 to 0 to 90 to 0% relative humidity (RH), in steps of 10% RH). GVS profiles were recorded at ambient temperature.

Abbreviations used in the experimental section:

Aq=aqueous

is DCM=dichloromethane

DMF=dimethylformamide

EtOAc=ethyl acetate

EtOH=ethanol

GVS=Gravimetric vapour sorption

MeOH=methanol

RT=RT

Rt=retention time

THF=tetrahydrofuran

Satd=saturated

Muscarinic antagonists, and the intermediates used in their preparation, described herein have been given IUPAC names generated by the Autonom 2000 plug in for IsisDraw version 2.5, as supplied by MDL Information Systems. Inc.

Intermediates Used in Preparation of Muscarinic Antagonists

The following intermediates 1-16 used in the preparation of muscarinic antagonists were prepared as follows:

### Intermediate 1: 2-Oxo-2-phenyl-N-prop-2-ynyl-acetamide

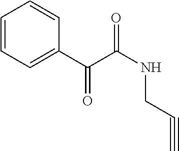

Oxalyl chloride (6.1 g, 48 mmol) was added to a solution of phenylglyoxylic acid (6.0 g, 40 mmol) and 3 drops of DMF in dry DCM (50 mL). The reaction mixture was stirred at RT for 3 h then the solvent was removed. The residue was taken up in dry DCM (50 mL) and the solution was cooled to 0° C. A mixture of propargyl amine (2.2 g, 40 mmol) and triethylamine (4.05 g, 40 mmol) was added cautiously over a period of 10 min then the mixture was allowed to warm to RT. Stirring was continued for 2.5 h then water (10 mL) was added. The mixture was washed with 1 M HCl, sat. sodium hydrogencarbonate (aq.), then brine. The organic phase was then dried (Na₂SO₄) and the solvent was removed. The residue was crystallized from cyclohexane to afford the product as a light brown solid.

Yield: 5.75 g, 76%. LC-MS (Method 3): Rt 2.47 min, m/z 188 [MH⁺].

### Intermediate 2: (5-Methyl-oxazol-2-yl)-phenyl-methanone

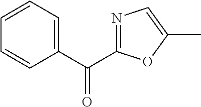

Methane sulfonic acid (10 g, 104 mmol) was added drop wise to a solution of 2-oxo-2-phenyl-N-prop-2-ynyl-acetamide (Intermediate 1) (2.4 g, 12.83 mmol) in 1,4-dioxane (20 mL). The resultant solution was heated at 90° C. for 66 h. The reaction mixture was cooled and the solvent was removed. The dark residue was partitioned between DCM and water. The DCM fraction was washed with 1 M HCl (2×), satd. sodium hydrogencarbonate solution (aq., 2×), then brine. The solution was dried (Na₂SO₄) and the solvent was removed to give the crude product. Purification was achieved via column chromatography, eluting with cyclohexane/EtOAc (4:1). This afforded the product as an off-white solid.

Yield: 1.0 g, 41%. LC-MS (Method 3): Rt 2.94 min, m/z 188 [MH⁺].

### Intermediate 3: (5-Bromomethyl-oxazol-2-yl)-phenyl-methanone

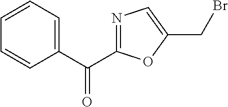

A mixture of (5-methyl-oxazol-2-yl)-phenyl-methanone (Intermediate 2) (0.8 g, 4.28 mmol), N-bromo-succinimide (0.9 g, 5.06 mmol) and 2,2′-azobis(2-methylpropionitrile) (56 mg, 0.34 mmol) in carbon tetrachloride (8 mL) was heated at reflux for 1.5 h. The reaction mixture was cooled to RT and filtered. The filtrate was diluted with DCM and washed with water, satd. sodium hydrogen carbonate solution (aq.) and brine. It was dried (Na₂SO₄) and the solvent was removed. Purification was achieved via column chromatography eluting with cyclohexane/EtOAc (4:1). This afforded the product as a yellow solid.

Yield: 0.9 g, 79%. LC-MS (Method 3): Rt 3.26 min, m/z 266, 268 [MH⁺].

### Intermediate 4: (5-Dimethylaminomethyl-oxazol-2-yl)-phenyl-methanone

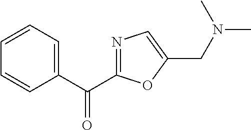

(5-Bromomethyl-oxazol-2-yl)-phenyl-methanone (Intermediate 3) (0.18 g, 0.68 mmol) was dissolved in a 2 M solution of dimethylamine in THF (3 mL, 6 mmol). The mixture was stirred at RT for 1 h with a precipitate forming almost instantly. The solvent was removed and the residue was partitioned between DCM and satd. sodium hydrogencarbonate solution (aq.). The aqueous phase was extracted with DCM and the combined organic phase was dried (Na₂SO₄) and the solvent removed to give the product as an orange oil that crystallized on standing.

Yield: 0.16 g, 99%. LC-MS (Method 2): Rt 1.22 min, m/z 231 [MH⁺].

### Intermediate 5: Cyclohexyl-(5-methyl-oxazol-2-yl)-phenyl-methanol

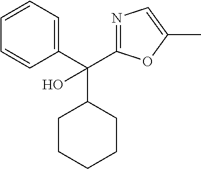

A solution of (5-methyl-oxazol-2-yl)-phenyl-methanone (intermediate 2) (3.0 g, 16 mmol) in 32 mL dry THF at 0° C. under nitrogen was treated dropwise over 10 min with a 2 M solution of cyclohexylmagnesium chloride in diethyl ether (10 mL, 20 mmol). The resulting deep yellow solution was stirred at 0° C. for about 30 min during which time a precipitate was formed, and then at RT for 1.5 h. The reaction mixture was cooled to 0° C. again and treated cautiously with satd. ammonium chloride solution (aq.). The mixture was stirred at RT for 10 min then diluted with water (10 mL). The phases were separated and the organic phase was washed with brine. The combined aqueous phase was extracted with DCM and the combined organic phase was dried (MgSO₄) and concentrated in vacuo to give the crude product, which was triturated with ether, filtered off and dried.

Yield: 3.65 g, 84%. LCMS (Method 3): Rt 3.78 min, m/z 272 [MH⁺].

### Intermediate 6: (5-Bromomethyl-oxazol-2-yl)-cyclohexyl-phenyl-methanol

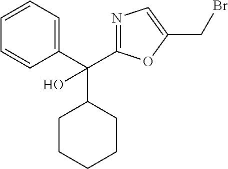

A solution of cyclohexyl-(5-methyl-oxazol-2-yl)-phenyl-methanol (Intermediate 5) (3.0 g, 11.1 mmol) in 1,2-dichloroethane (22 mL) was treated with N-bromo-succinimide (2.16 g, 12.2 mmol) followed by 2,2′-azobis(2-methylpropionitrile) (0.18 g, 2.1 mmol). The mixture was heated to 80° C. for 2.5 h and then allowed to cool to RT. Satd. sodium hydrogen carbonate solution (aq.) was added and the phases were separated. The organic layer was washed with brine and the combined aqueous layers were extracted with DCM.

The combined organic phase was dried (MgSO₄) and concentrated in vacuo to give the crude product as a brown oil. Purification was achieved via column chromatography eluting with 33-100% DCM/cyclohexane, followed by 25% EtOAc/DCM.

Yield: 1.85 g, 48%. LCMS (Method 3): Rt 4.27 min, m/z 350, 352 [MH⁺].

### Intermediate 7: 2-Phenethyloxy-ethanol

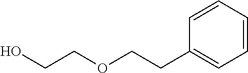

The preparation of 2-phenethyloxy-ethanol is described in J. Med. Chem. 1983, 26, 1570-1576.

### Intermediate 8: [2-(2-Bromo-ethoxy)-ethyl]-benzene

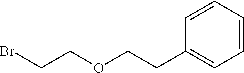

Triphenyl phosphine (1.65 g, 6.3 mmol) was added to a solution of 2-phenethyloxy-ethanol (Intermediate 7) (950 mg, 5.7 mmol) and carbon tetrabromide (2.09 g, 6.3 mmol) in DCM (25 mL) and stirred at RT for 6 h. Then a further equivalent of triphenyl phosphine and carbon tetrabromide was added and stirred overnight. The reaction mixture was concentrated and the residue was purified by column chromatography over silica using cyclohexane as eluent. Concentration of the pure fractions afforded the product as a clear oil.

Yield: 1.25 g, 96%.

¹H NMR (CDCl₃): δ2.91 (t, 2H), 3.44 (t, 2H), 3.71 (t, 2H), 3.76 (t, 2H), 7.19-7.24 (m, 3H), 7.27-7.31 (m, 2H) ppm.

### Intermediate 9: 2-(4-Methyl-benzyloxy)-ethanol

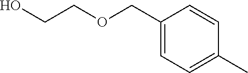

A mixture of potassium hydroxide (1.19 g, 21.3 mmol) in ethylene glycol (12 mL, 213 mmol) was heated at 130° C. for 3 h, then cooled to 35° C., and 4-methylbenzyl bromide (3.94 g, 21.3 mmol) was added. The reaction mixture was heated at 35° C. for 20 h, cooled to RT, and partitioned between water and diethyl ether. The aqueous layer was extracted with diethyl ether. The combined organic layers were washed with brine, dried (MgSO₄), and concentrated to dryness to afford a brown oil. This was purified by column chromatography over silica using a gradient of 0-100% diethyl ether/cyclohexane. The pure fractions were combined and concentrated to afford a yellow liquid.

Yield: 2.97 g, 84%.

¹H NMR (CDCl₃): δ2.04 (t, 1H), 2.35 (s, 3H), 3.58 (t, 2H), 3.75 (m, 2H), 4.52 (s, 2H), 7.16 (d, 2H), 7.23 (d, 2H) ppm.

### Intermediate 10:1-(2-Bromo-ethoxymethyl)-4-methyl-benzene

Prepared analogous to the method used for Intermediate 8, but using 1-(2-bromo-ethoxymethyl)-4-methyl-benzene (Intermediate 9) instead of 2-phenethyloxy-ethanol (Intermediate 9) was:

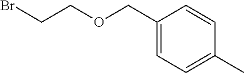

Yield: 85%.

¹H NMR (CDCl₃): δ2.35 (s, 3H), 3.47 (t, 2H), 3.76 (t, 2H), 4.55 (s, 2H), 7.16 (d, 2H), 7.24 (d, 2H) ppm.

### Intermediate 11: 4-(3-Bromo-propoxy)-1,2-dichloro-benzene

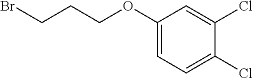

A mixture of 3,4-dichlorophenol (1.98 g, 12.14 mmol), 1,3-dibromopropane (6.0 mL, 59 mmol, and potassium carbonate (2.5 g, 18 mmol) in acetonitrile was heated at 80° C. overnight. The reaction mixture was cooled to RT, filtered, and the filtrate partitioned between water and diethyl ether. The organic layer was dried (MgSO₄), concentrated, and purified by column chromatography over silica using 0-10% diethyl ether/cyclohexane as eluent to afford the product.

Yield: 2.96 g, 86%.

¹H NMR (CDCl₃): δ2.32 (m, 2H), 3.59 (t, 2H), 4.08 (t, 2H), 6.77 (dd, 1H), 7.00 (d, 1H), 7.32 (d, 1H) ppm.

### Intermediate 12: 2-(3,4-Dichloro-benzyloxy)-ethanol

Prepared analogous to the method used for Intermediate 9, but using 3,4-dichlorobenzyl chloride instead of 4-methylbenzyl bromide was:

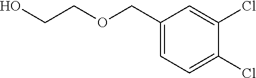

Yield: 72%.

¹H NMR (CDCl₃): δ1.83 (br.s, 1H), 3.61 (t, 2H), 3.79 (t, 2H), 4.52 (s, 2H), 7.17 (dd, 1H), 7.42 (d, 1H), 7.45 (d, 1H) ppm.

### Intermediate 13: 4-(2-Bromo-ethoxymethyl)-1,2-dichloro-benzene

Prepared analogous to the method used for Intermediate 8, but using 2-(3,4-dichloro-benzyloxy)-ethanol (Intermediate 12) instead of 2-phenethyloxy-ethanol (Intermediate 7) was:

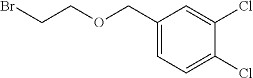

Yield: quantitative.

¹H NMR (CDCl₃): δ3.50 (t, 2H), 3.80 (t, 2H), 4.53 (s, 2H), 7.19 (dd, 1H), 7.42 (d, 1H), 7.46 (d, 1H) ppm.

### Intermediate 14: Methanesulfonic acid 2-(4-chloro-benzyloxy)-ethyl ester

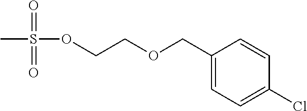

A solution of methanesulfonyl chloride (980 μL, 12.6 mmol) in dry DCM (10 mL) was slowly added to a cooled (0° C.) solution of 2-(4-chloro-benzyloxy)-ethanol (2.14 g, 11.46 mmol) and diisopropyethylamine (2.0 mL, 23 mmol) in dry DCM (10 mL). The reaction mixture was allowed to warm to RT overnight. Water was added and the organic layer was dried (MgSO₄) and concentrated. The residue was purified by column chromatography over silica using a gradient of 0-20% diethyl ether/cyclohexane to afford the pure product.

Yield: 1.87 g, 67%.

¹H NMR (CDCl₃): δ3.03 (s, 3H), 3.74 (m, 2H), 4.39 (m, 2H), 4.54 (s, 2H), 7.27 (d, 2H), 7.33 (d, 2H) ppm.

### Intermediate 15: 1-(2-Bromo-ethoxymethyl)-4-chloro-benzene

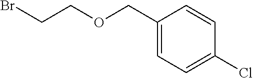

A mixture of methanesulfonic acid 2-(4-chloro-benzyloxy)-ethyl ester (Intermediate 14) (1.37 g, 5.18 mmol) and lithium bromide (1.80 g, 20.7 mmol) in acetone (15 mL) was heated at reflux overnight. The reaction mixture was concentrated to dryness and the residue partitioned between DCM and water. The organic layer was dried (MgSO₄), and concentrated, and purified by column chromatography over silica using DCM/cyclohexane (1:3) as eluent to afford the product as a colourless oil.

Yield: 0.67 g, 78%.

¹H NMR (CDCl₃): δ3.49 (t, 2H), 3.79 (t, 2H), 4.55 (s, 2H), 7.30 (d, 2H), 7.32 (d, 2H) ppm.

### Intermediate 16: Cyclohexyl-(5-dimethylaminomethyl-oxazol-2-yl)-phenyl-methanol

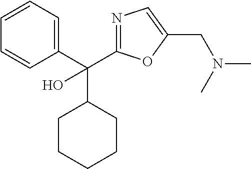

A solution of (5-bromomethyl-oxazol-2-yl)-cyclohexyl-phenyl-methanol (Intermediate 6) (3.2 g, 9.2 mmol) in THF (40 mL) was treated with a 2 M solution of dimethylamine in THF (40 mL, 80 mmol). A suspension formed after stirring for a few minutes. The reaction mixture was left at RT overnight and then the solid was filtered off and discarded. The filtrate was concentrated under reduced pressure and the residue was partitioned between

DCM and satd. sodium hydrogen carbonate solution (aq.). The organic layer was dried (Na₂SO₄) and evaporated to afford the title compound as a solid.

Yield: 2.74 g, 95%.

LC-MS (Method 1): Rt 6.57 min, m/z 315 [MH⁺].

¹H NMR (DMSO-d₆): δ0.92-1.29 (m, 6H), 1.42-1.74 (m, 4H), 2.10 (s, 6H), 2.22 (m, 1H), 3.45 (s, 2H), 5.90 (s, 1H), 6.98 (s, 1H), 7.18-7.22 (m, 1H), 7.27-7.34 (m, 2H), 7.40-7.46 (m, 2H) ppm.

The two enantiomers of cyclohexyl-(5-dimethylaminomethyl-oxazol-2-yl)-phenyl-methanol (Intermediate 16) (2.74 g) were separated by preparative chiral HPLC using a 250×20 mm Chiralpak® IA column packed with amylase tris(3,5-dimethylphenyl-carbamate) immobilized on 5 μm silica gel. The column was eluted with 5% EtOH in heptane buffered with 0.1% diethylamine at 15 mL/min. The first eluting enantiomer (Rt 8.5 min) afforded (S)-cyclohexyl-(5-dimethylaminomethyl-oxazol-2-yl)-phenyl-methanol (Intermediate 16a) as a white solid.

### Intermediate 16a: (S)-cyclohexyl-(5-dimethylaminomethyl-oxazol-2-yl)-phenyl-methanol

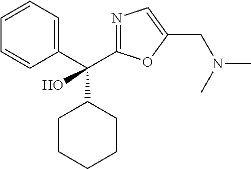

Yield: 0.73 g, 27%.

LC-MS (Method 1): Rt 6.50 min, m/z 315 [MH⁺].

¹H NMR (CDCl₃): δ1.12-1.39 (m, 7H), 1.62-1.76 (m, 3H), 2.25 (s, 6H), 2.29-2.32 (m, 1H), 3.54 (dd_{AB}, 2H), 3.70 (br.s, 1H), 6.84 (s, 1H), 7.24 (t, 1H), 7.33 (t, 2H), 7.64 (d, 2H) ppm.

The second eluting enantiomer (Rt 10.3 min) afforded (R)-cyclohexyl-(5-dimethylaminomethyl-oxazol-2-yl)-phenyl-methanol (Intermediate 16b) as a white solid.

### Intermediate 16b: (R)-cyclohexyl-(5-dimethylaminomethyl-oxazol-2-yl)-phenyl-methanol

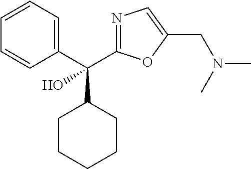

Yield: 1.04 g, 38%.

LC-MS (Method 1): Rt 6.48 min, m/z 315 [MH⁺].

¹H NMR (CDCl₃): δ1.10-1.39 (m, 7H), 1.62-1.76 (m, 3H), 2.25 (s, 6H), 2.29-2.35 (m, 1H), 3.54 (dd_{AB}, 2H), 3.70 (br.s, 1H), 6.84 (s, 1H), 7.24 (t, 1H), 7.33 (t, 2H), 7.64 (d, 2H) ppm.

### Muscarinic Antagonist 1 (MA1): [2-((S)-Cyclohexyl-hydroxy-phenyl-methyl)-oxazol-5-ylmethyl]-dimethyl-(3-phenoxy-propyl)-ammonium bromide

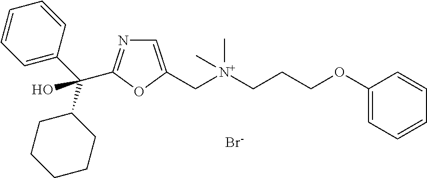

A solution of (S)-cyclohexyl-(5-dimethylaminomethyloxazol-2-yl)-phenyl-methanol (Intermediate 16a) (0.060 g, 0.19 mmol) and 3-phenoxypropyl bromide (0.215 g, 1 mmol) in acetonitrile (1.33 mL) and chloroform (2 mL) was allowed to stand at RT for 5 days. The solvent was removed to afford the crude product. Purification was achieved by column chromatography eluting sequentially with DCM, 2.5%, 5%, 10% then 20% MeOH in DCM.

Yield: 50 mg, 43%.

LC-MS (Method 1): Rt 8.32 min, m/z 449 [M⁺].

¹H NMR (CDCl₃): δ1.06-1.17 (m, 3H), 1.23-1.36 (m, 4H), 1.52-1.85 (m, 3H), 2.28-2.35 (m, 3H), 3.32 (s, 3H), 3.33 (s, 3H), 3.63 (dd, 2H), 4.04 (t, 2H), 5.23 (dd_{AB}, 2H), 6.85 (d, 2H), 6.98 (t, 1H), 7.20 (t, 1H), 7.26-7.30 (m, 4H), 7.55-7.58 (m, 3H) ppm.

### Muscarinic Antagonist 2 (MA2): [2-((R)-Cyclohexyl-hydroxy-phenyl-methyl)-oxazol-5-ylmethyl]-dimethyl-(3-phenoxy-propyl)-ammonium bromide

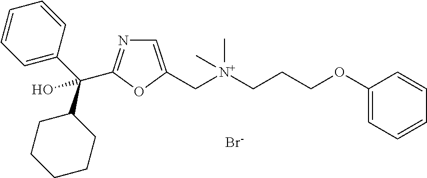

A solution of (R)-cyclohexyl-(5-dimethylaminomethyloxazol-2-yl)-phenyl-methanol (Intermediate 16b) (98 mg, 0.31 mg) and 3-phenoxypropyl bromide (740 mg, 3.44 mmol) in chloroform (1.5 mL) and acetonitrile (1.5 mL) was heated at 50° C. for 22 h. The RM was concentrated to dryness to afford a colourless viscous oil, which was triturated with diethyl ether to furnish a white gum. This was purified by column chromatography eluting with 2.5-25% MeOH/DCM to afford the product as a turbid viscous oil. Drying under vacuum at 45° C. for 1-2 days afforded a white solid.

Yield: 142 mg, 86%.

LC-MS (Method 1): Rt 8.41 min, m/z 449 [M⁺].

¹H NMR (CDCl₃): δ1.06-1.16 (m, 3H), 1.21-1.37 (m, 4H), 1.59-1.74 (m, 3H), 2.32 (m, 3H), 3.32 (s, 3H), 3.33 (s, 3H), 3.61 (dd, 2H), 4.03 (t, 2H), 4.14 (br.s, 1H), 5.20 (dd_{AB}, 2H), 6.85 (d, 2H), 6.98 (t, 1H), 7.19 (t, 1H), 7.26-7.30 (m, 4H), 7.55-7.58 (m, 3H) ppm.

### Muscarinic Antagonist 2 (MA2): [2-((R)-Cyclohexyl-hydroxy-phenyl-methyl)-oxazol-5-ylmethyl]-dimethyl-(3-phenoxy-propyl)-ammonium bromide—Crystalline Form A

General Experimental conditions for the preparation of MA2 Crystalline Form A are the same as those described herein below in Preparation [2] of MA 11.

### (R)-cyclohexyl-(5-dimethylaminomethyl-oxazol-2-yl)-phenyl-methanol¹

(5-Dimethylaminomethyl-oxazo-2-yl)-phenyl-methanone² was dissolved in THF (8.4 L/kg) and cooled to a temperature of 0±5° C., to which cyclohexyl magnesium chloride (1.3 eq., as a 20 w/w % solution in Toluene/THF) was dosed over at least 1 h. The reaction mixture was heated to 20° C. over 40 min and stirred at 20° C. for at least 1 h, at which point conversion to product was >96% by HPLC. The reaction mixture was dosed to a mixture of 23.1 w/w % NH₄Cl (3.97 L/kg) and water (3.97 L/kg). The phases were separated and the aqueous layer extracted with ethyl acetate (7 L/kg). The combined organic layers were washed with water (5.25 L/kg), and 70% of the volume removed by distillation (p≧130 mbar, 50° C.). To the distillation residue acetonitrile (7.82 L/kg) was added and the suspension heated until complete dissolution was attained (70° C.). The reaction was then cooled to 0° C. over 7 h and stirred at 0° C. for at least 1 h. The reaction product (±)-cyclohexyl-(5-dimethylaminomethyl-oxazol-2-yl)-phenyl-methanol was then collected by filtration and washed three times with cold acetonitrile (1.65 L/kg). Yields achieved with this procedure ranged between 60-70% and the purities achieved were >97% peak area (HPLC) and >97% w/w (NMR). R)-Cyclohexyl-(5-dimethylaminomethyl-oxazol-2-yl)-phenyl-methanol was separated from this racemic mixture by chiral SMB chromatography on a Chiralpak AD column using acetonitrile: isopropanol: diethylmethylamine (90:10:0.1) as eluent. ¹An alternative preparation of (R)-cyclohexyl-(5-dimethylaminomethyl-oxazol-2-yl)-phenyl-methanol is described in WO 2007/017669 (example 6).²A preparation of (5-dimethylaminomethyl-oxazo-2-yl)-phenyl-methanone is described in WO 2007/017669 (intermediate 4).

Procedure for Preparing Seed Crystals of (MA2) Crystalline Form A—Procedure 1

(R)-cyclohexyl-(5-dimethylaminomethyl-oxazol-2-yl)-phenyl-methanol (leg) and 3-phenoxypropyl bromide (1.1 eq) were suspended in iso-propanol (4.3 L/kg). The resulting suspension was heated at reflux for at least 20 h, or until the conversion to product was >98% by HPLC. The resulting solution was diluted with iso-propanol (2 L/kg) and cooled to 50° C. At 50° C., tert-butyl methyl ether (TBME) (9.5 L/kg) was added and the solution stirred at 50° C. for a further 2 h during which time spontaneous crystallization occurred. The mixture was gradually cooled to 0° C. over a 3 h period and stirred at 0° C. for at least 1 h. The crystalline product was collected by filtration and washed four times with cold TBME (0.16 L/kg). Product yields with this procedure were >80% and the purity was >98% peak area (HPLC) and >97% w/w (NMR).

Procedure for Preparing Seed Crystals of (MA2) Crystalline Form A—Procedure 2

(R)-cyclohexyl-(5-dimethylaminomethyl-oxazol-2-yl)-phenyl-methanol (1 eq) and 3-phenoxypropyl bromide (1.1 eq) were slurried in iso-propanol (3.14 L/kg). The resulting suspension was heated to reflux (100° C.), at which complete dissolution was achieved. After heating at reflux for 8½ h the reaction mixture was cooled to ambient temperature over night. Analysis by HPLC showed complete conversion to product. A sample of the reaction mixture (0.043 L/kg) was withdrawn and TBME (0.14 L/kg) added dropwise whereupon precipitation occurred. This suspension was charged to the reaction mixture at ambient temperature whereupon crystallization occurred. The resulting suspension was cooled to 0° C. and stirred for 3 h at this temperature. The product was collected by filtration, using iso-propanol (2.14 L/kg) to aid transfer from vessel to filter. The filter cake was washed with iso-propanol (1 L/kg) and dried on a rotary evaporator over night. The crude product was obtained as a white solid in 86% yield.

The crude product was charged in TBME (10.4 L/kg with respect to crude product) and stirred at ambient temperature for 2 h. The product was collected by filtration, and the filter cake washed with TBME (20 ml), and dried on a rotary evaporator overnight. Yield was 94% from crude product, and purity 98.3% peak area by HPLC.

Preparation of (MA2) Crystalline Form A

To (R)-cyclohexyl-(5-dimethylaminomethyl-oxazol-2-yl)-phenyl-methanol (1 eq) in iso-propanol (4.44 L/kg) at ambient temperature was added 3-phenoxypropyl bromide (1.1 eq). The mixture was heated to reflux temperature (83° C.) over 90 min. and stirred at reflux for 20 h. After that time the mixture was cooled to 57° C. over 13 min. A sample was taken and then the reaction mixture was heated again to reflux. Reaction conversion was determined to be 98.4% by HPLC.

The reaction mixture was diluted with iso-propanol (5.55 L/kg) and cooled to 57° C. The solution was filtered through a heated in line filter into a stirring vessel. The reactor and the filter lines were rinsed with warm (55° C.) iso-propanol (1.11 L/kg). The content of the stirring vessel was transferred back into the reactor and rinsed with iso-propanol (1.11 L/kg). Iso-propanol (5.55 L/kg) was distilled off at a temperature of 47° C.-50° C. and a pressure of 200 mbar. The residue was cooled to 52° C. At this temperature TBME (10 L/kg) was added over 35 min. The resulting solution was stirred for 2 h at 50° C. Seed crystals (1.18% w/w (with respect to input (R)-cyclohexyl-(5-dimethylaminomethyl-oxazol-2-yl)-phenyl-methanol)) were added and the mixture was stirred for additional 2 h at 50° C. The suspension formed was cooled to 0° C. over 3 h and stirred at that temperature for 13 h. After filtration the filter cake was rinsed four times with 1° C.-8° C. cold TBME (1.48 L/kg, 1.67 L/kg, 2.04 L/kg and 2.04 L/kg). The filter cake was pre-dried for 4.5 hr in a stream of nitrogen and afterwards it was further dried on a rotary evaporator at 45° C. and ≧12 mmbar to yield the product as a crystalline white solid. Yield obtained by this process on a 2.7 kg scale was 90.5% and the purity 98.3% peak area (HPLC) and 98.9% w/w (NMR). Loss on drying was 0.23% w/w (gravimetric).

Analysis of Muscarinic Antagonist 2 (MA2) Crystalline Form A

A sample of crystalline Form A obtained by the ‘Procedure for Preparing Seed Crystals of Crystalline Form A—Procedure 2’ was analysed by XRPD, DSC and TGA.

The melting temperature of Form A as determined by DSC was found to be 150° C. (onset) (±2° C.). Weight loss observed prior to melting by TGA was negligible, near 0.0%. GVS determination gave a 0.8% weight increase (% w/w) at 80% RH (±0.2%).

An XRPD spectrum of Muscarinic Antagonist 2 (MA2) Crystalline Form A is presented in FIG. 1.

### Muscarinic Antagonist 3 (MA3): [2-((R)-Cyclohexyl-hydroxy-phenyl-methyl)-oxazol-5-ylmethyl]-dimethyl-(2-phenethyloxy-ethyl)-ammonium bromide

Prepared according to the method used in preparing MA2, but using [2-(2-bromo-ethoxy)-ethyl]-benzene (Intermediate 10) instead of 3-phenoxypropyl bromide.

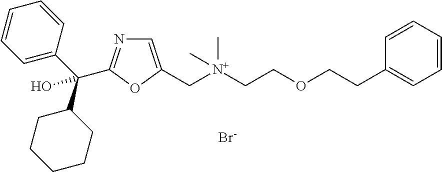

Yield: 94%.

LC-MS (Method 1): Rt 8.50 min, m/z 463 [M⁺].

¹H NMR (CD₃OD): δ1.06-1.39 (m, 6H), 1.55 (m, 1H), 1.65-1.79 (m, 3H), 2.40 (m, 1H), 2.90 (t, 2H), 2.94 (s, 6H), 3.47 (m, 2H), 3.78 (t, 2H), 3.86 (m, 2H), 4.56 (s, 2H), 7.12 (m, 1H), 7.19-7.28 (m, 5H), 7.32-7.37 (m, 3H), 7.55 (m, 2H).

### Muscarinic Antagonist 4 (MA4): [2-((R)-Cyclohexyl-hydroxy-phenyl-methyl)-oxazol-5-ylmethyl]-[3-(3,4-dichloro-phenoxy)-propyl]-dimethyl-ammonium bromide

Prepared according to the method used in MA2, but using 4-(3-bromo-propoxy)-1,2-dichloro-benzene (Intermediate 11) instead of 3-phenoxypropyl bromide.

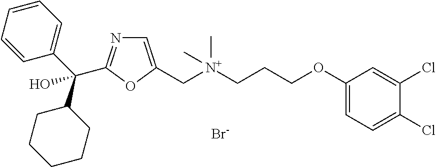

Yield: 59%.

LC-MS (Method 4): Rt 8.85 min, m/z 517 [M⁺].

¹H NMR (CDCl₃): δ 1.08-1.40 (m, 7H), 1.60-1.76 (m, 3H), 2.34 (m, 3H), 3.34 (s, 6H), 3.65 (m, 2H), 3.99 (m, 3H), 5.25 (dd_{AB}, 2H), 6.73 (dd, 1H), 6.96 (d, 1H), 7.22 (t, 1H), 7.26-7.34 (m, 3H), 7.56 (m, 3H) ppm

### Muscarinic Antagonist 5 (MA5): [2-((R)-Cyclohexyl-hydroxy-phenyl-methyl)-oxazol-5-ylmethyl]-[2-(3,4-dichloro-benzyloxy)-ethyl]-dimethyl-ammonium bromide

Prepared according to the method used in MA2, but using 4-(2-bromo-ethoxymethyl)-1,2-dichloro-benzene (Intermediate 13) instead of 3-phenoxypropyl bromide.

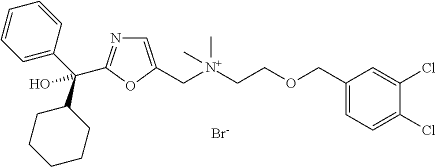

Yield: 86%.

LC-MS (Method 1): Rt 9.07 min, m/z 517 [M⁺].

¹H NMR (CDCl₃): δ 1.09-1.37 (m, 7H), 1.60-1.77 (m, 3H), 2.31 (m, 1H), 3.33 (s, 6H), 3.91 (m, 2H), 3.98 (m, 3H), 4.55 (s, 2H), 5.20 (dd_{AB}, 2H), 7.17 (dd, 1H), 7.24 (m, 1H), 7.31 (t, 2H), 7.40 (d, 1H), 7.44, (d, 1H), 7.48, (s, 1H), 7.56 (d, 2H) ppm.

### Muscarinic Antagonist 6 (MA6): [2-(4-chloro-benzyloxy)-ethyl]-[2-((R)-cyclohexyl-hydroxy-phenyl-methyl)-oxazol-5-ylmethyl]-dimethyl-ammonium bromide

Prepared according to the method used in MA2, but using 1-(2-bromo-ethoxymethyl)-4-chloro-benzene (Intermediate 15) instead of 3-phenoxypropyl bromide.

A solution of (R)-cyclohexyl-(5-dimethylaminomethyloxazol-2-yl)-phenyl-methanol (0.40 g, 1.27 mmol) and 1-(2-bromo-ethoxymethyl)-4-chloro-benzene (Intermediate 15) (0.67 g, 2.68 mmol) in chloroform (4 mL) and acetonitrile (4 mL) was heated at 50° C. for 3 days. The reaction mixture was concentrated to dryness to afford a yellow oil, which was purified by column chromatography eluting with 2.5-25% MeOH/DCM to afford the product as a white foam. Yield, 0.68 g, 92%

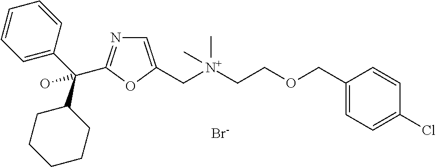

Yield: 92%.

LC-MS (Method 1): Rt 8.72 min, m/z 483 [M⁺].

¹H NMR (CDCl₃): δ 1.08-1.40 (m, 7H), 1.61-1.76 (m, 3H), 2.31 (m, 1H), 3.32 (s, 6H), 3.88 (m, 2H), 3.94 (m, 2H), 4.03 (br. s, 1H), 4.54 (s, 2H), 5.17 (dd_{AB}, 2H), 7.21-7.26 (m, 3H), 7.28-7.34 (m, 4H), 7.46 (s, 1H), 7.56 (d, 2H) ppm.

### Muscarinic Antagonist 7 (MA7): [2-((R)-Cyclohexyl-hydroxy-phenyl-methyl)-oxazol-5-ylmethyl]-dimethyl-(3-phenoxy-propyl)-ammonium hemi-naphthalene-1,5-disulfonate

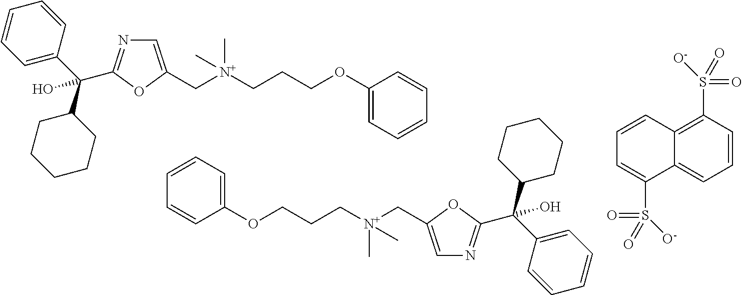

A mixture of [2-((R)-cyclohexyl-hydroxy-phenyl-methyl)-oxazol-5-ylmethyl]-dimethyl-(3-phenoxy-propyl)-ammonium bromide (MA2) (201 mg, 0.372 mmol), naphthalene-1,5-disulfonate disodium salt (68 mg, 0.21 mmol), DCM (2.8 mL), and water (2.8 mL) was stirred vigorously at RT overnight. The solids were collected by filtration, washed with DCM/water mixture, and dried under vacuum at 40° C. The sample of MA7 obtained is hereinafter referred to as the MA7 Amorphous Form

¹H NMR showed a spectrum corresponding to the hemi-salt (2:1 ratio of cation/anion).

Yield: 208 mg, 94%.

LC-MS (Method 1): Rt 8.35 min, m/z 449 [M+].

¹H NMR (CD₃OD): δ 1.04-1.37 (m, 12H), 1.55-1.75 (m, 8H), 2.22 (m, 4H), 2.40 (m, 2H), 3.01 (s, 6H), 3.02 (s, 6H), 3.37 (m, 2H), 3.97 (m, 4H), 4.67 (s, 4H), 6.89 (d, 4H), 6.95 (t, 2H), 7.21 (t, 2H), 7.28 (m, 8H), 7.51 (m, 8H), 8.19 (d, 2H), 9.02 (d, 2H) ppm.

Salt Form 1

MA7 Amorphous form (as prepared herein above) was heated in toluene with stirring at 60° for 48 hours and allowed to cool to RT while stirring to afford the product as small platelets. The product was collected by filtration and dried under vacuum at 50° C. for 3 h. The melting temperature of Form 1 was determined by DSC, during which testing Form 1 underwent dehydration and subsequently the dehydrated Form 1, totally or partially converted into an anhydrous form, melted at 225° C.±2° C. (onset). Water content as determined by TGA was 0.7% (±0.2%). GVS determination gave a 3.1% weight increase (% w/w) at 80% RH (±0.5%).

An XRPD spectrum of Form 1 is presented in FIG. 2.

Further quantities of Form 1 were prepared as follows: MA7 Amorphous form was crystallised from refluxing acetonitrile using a hot filtration of the solution and allowed to cool to RT while stirring to afford the product as small platelets. The product was collected by filtration and stirred in toluene at 60° C. for 19 h. The solids were collected by decanting the solvent and dried under vacuum at 50° C. for 3 h. XRPD and DSC analysis were consistent with Form 1.

Salt Form 2

MA7 Amorphous form was heated in anisole at 154° C. for 3 hrs then left to stand at RT for 48 hrs. The solids were collected by decanting the solvent and dried under vacuum at 45° C. The melting temperature of Form 2 as determined by DSC was found to be 227° C.±2° C. (onset). Water content as determined by TGA was 0.0%. GVS determination gave a 0.7% weight increase (% w/w) at 80% RH (±0.2%).

An XRPD spectrum of Form 2 is presented in FIG. 3.

Further quantities of Form 2 were prepared as follows: MA7 Amorphous form was crystallised from refluxing chlorobenzene and allowed to slowly cool to RT to afford the product as fine needles. The product was collected by filtration and dried under vacuum at RT overnight. XRPD and DSC analysis were consistent with Form 2.

Further quantities of Form 2 were prepared as follows: MA7 Amorphous form was stirred in toluene at 80° C. over for at least 60 hours. The solids were collected by decanting the solvent and dried under vacuum at 45° C. XRPD and DSC analysis were consistent with Form 2.

Salt Form 3

MA7 Amorphous form was crystallised from refluxing acetone/water mixture using a hot filtration of the solution and allowed to cool to RT while stirring to afford the product as a white powder. The product was collected by filtration and dried under vacuum at RT overnight.

The melting temperature of Form 3 was determined by DSC, during which testing Form 3 underwent dehydration and subsequently the dehydrated Form 3, totally or partially converted into an anhydrous form, melted at 224° C.±2° C. (onset). Water content as determined by TGA was 2.1% (±0.2%). GVS determination gave a 3.0% weight increase (% w/w) at 80% RH (±0.2%).

An XRPD spectrum of Form 3 is presented in FIG. 4.

### Muscarinic Antagonist 8 (MA8): [2-((R)-Cyclohexyl-hydroxy-phenyl-methyl)-oxazol-5-ylmethyl]-dimethyl-(2-phenethyloxy-ethyl)-ammonium hemi-naphthalene-1,5-disulfonate

Prepared according to the method used in MA7, but using [2-((R)-cyclohexyl-hydroxy-phenyl-methyl)-oxazol-5-ylmethyl]-dimethyl-(2-phenethyloxy-ethyl)-ammonium bromide (MA3) instead of [2-((R)-cyclohexyl-hydroxy-phenyl-methyl)-oxazol-5-ylmethyl]-dimethyl-(3-phenoxy-propyl)-ammonium bromide.

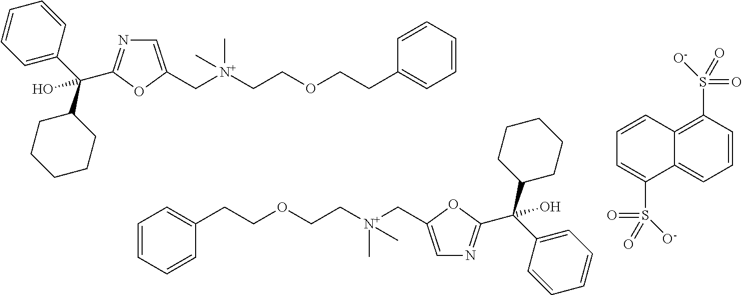

Yield: 98%.

LC-MS (Method 1): Rt 8.64 min, m/z 463 [M⁺].

¹H NMR (CD₃OD): δ 1.05-1.39 (m, 12H), 1.53 (m, 2H), 1.68 (m, 4H), 1.77 (m, 2H), 2.39 (m, 2H), 2.85 (s, 12H), 2.87 (t, 4H), 3.36 (m, 4H), 3.72 (t, 4H), 3.76 (m, 4H), 4.46 (s, 4H), 7.11 (m, 2H), 7.20 (m, 8H), 7.22-7.27 (m, 2H), 7.33 (t, 6H), 7.54 (m, 6H), 8.20 (dd, 2H), 9.02 (d, 2H) ppm.

Crystallised from refluxing acetonitrile and allowed to slowly cool to RT to afford the product as fine needles. Melting point: 215-216 (10° C./min).

### Muscarinic Antagonist 9 (MA9): [2-((R)-Cyclohexyl-hydroxy-phenyl-methyl)-oxazol-5-ylmethyl]-[3-(3,4-dichloro-phenoxy)-propyl]-dimethyl-ammonium hemi-naphthalene-1,5-disulfonate

Prepared according to the method used in MA7, but using [2-((R)-cyclohexyl-hydroxy-phenyl-methyl)-oxazol-5-ylmethyl]-[3-(3,4-dichloro-phenoxy)-propyl]-dimethyl-ammonium bromide (MA4) instead of [2-((R)-cyclohexyl-hydroxy-phenyl-methyl)-oxazol-5-ylmethyl]-dimethyl-(3-phenoxy-propyl)-ammonium bromide.

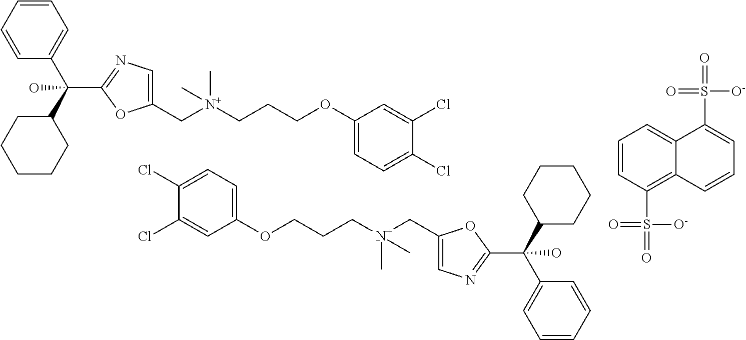

Yield: 56%.

LC-MS (Method 1): Rt 9.13 min, m/z 517 [M⁺].

¹H NMR (CD₃OD): δ 1.05-1.37 (m, 12H), 1.56-1.75 (m, 8H), 2.23 (m, 4H), 2.40 (m, 2H), 3.03 (s, 6H), 3.04 (s, 6H), 3.34 (m, 4H), 3.96 (m, 4H), 4.68 (s, 4H), 6.85 (dd, 2H), 7.09 (d, 2H), 7.21 (m, 2H), 7.30 (t, 4H), 7.42 (d, 2H), 7.52 (m, 8H), 8.20 (dd, 2H), 9.02 (dd, 2H) ppm.

Crystallised from hot MeOH. Melting point: 225-227° C. (1° C./min).

### Muscarinic Antagonist 10 (MA10): [2-((R)-Cyclohexyl-hydroxy-phenyl-methyl)-oxazol-5-ylmethyl]-[2-(3,4-dichloro-benzyloxy)-ethyl]-dimethyl-ammonium hemi-naphthalene-1,5-disulfonate

Prepared according to the method used in MA7, but using [2-((R)-cyclohexyl-hydroxy-phenyl-methyl)-oxazol-5-ylmethyl]-[2-(3,4-dichloro-benzyloxy)-ethyl]-dimethyl-ammonium bromide (MA5) instead of [2-((R)-cyclohexyl-hydroxy-phenyl-methyl)-oxazol-5-ylmethyl]-dimethyl-(3-phenoxy-propyl)-ammonium bromide.

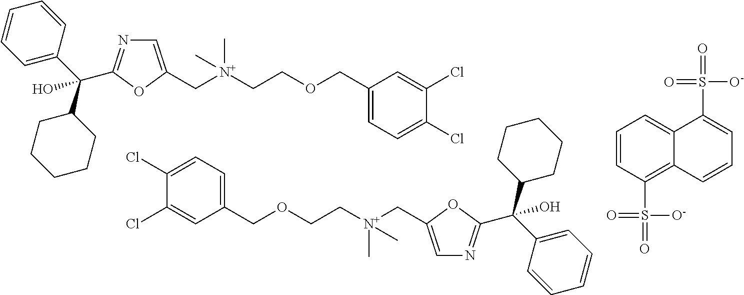

Yield: 76%. LC-MS (Method 1): Rt 9.06 min, m/z 517 [M⁺].

¹H NMR (CD₃OD): δ 1.05-1.37 (m, 12H), 1.54 (m, 2H), 1.63-1.76 (m, 6H), 2.38 (m, 2H), 3.03 (s, 12H), 3.47 (m, 4H), 3.86 (m, 4H), 4.51 (s, 4H), 4.71 (s, 4H), 7.22-7.33 (m, 8H), 7.46 (s, 2H), 7.52 (m, 10H), 8.20 (dd, 2H), 9.02 (d, 2H) ppm.

### Muscarinic Antagonist 11 (MA11): [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate

Preparation [1]

MA11 may be prepared according to the method used in MA7, but using [2-(4-chloro-benzyloxy)-ethyl]-[2-((R)-cyclohexyl-hydroxy-phenyl-methyl)-oxazol-5-ylmethyl]-dimethyl-ammonium bromide (MA6) instead of [2-((R)-cyclohexyl-hydroxy-phenyl-methyl)-oxazol-5-ylmethyl]-dimethyl-(3-phenoxy-propyl)-ammonium bromide. An example preparation is described below.

A mixture of [2-(4-chloro-benzyloxy)-ethyl]-[2-((R)-cyclohexyl-hydroxy-phenyl-methyl)-oxazol-5-ylmethyl]-dimethyl-ammonium bromide (0.20 g, 0.36 mmol), naphthalene-1,5-disulfonate disodium salt (0.059 g, 0.18 mmol), DCM (2.8 mL), and water (2.8 mL) was stirred vigorously at RT overnight. N-heptane (1.0 mL) was added and the mixture was stirred vigorously. On standing two clear layers and a yellow oil were obtained. DCM (1.0 mL) was added (causing the oil to dissolve) and the mixture was stirred at RT overnight resulting in precipitation of a white solid. The solid was collected by filtration, washed with DCM/water mixture, and dried under vacuum at 50° C.

¹H NMR showed a spectrum corresponding to the hemi-salt (2:1 ratio of cation/anion).

Yield: 0.17 g, 77%.

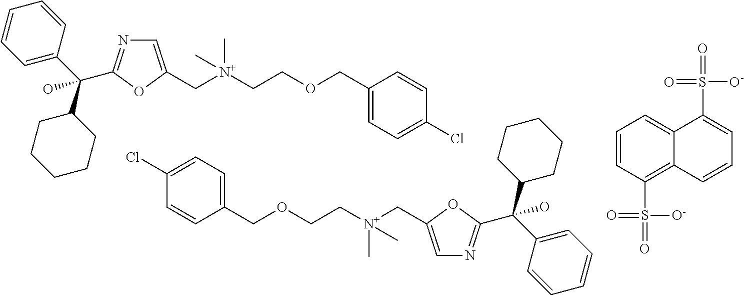

LC-MS (Method 1): Rt 8.62 min, m/z 483 [M⁺].

¹H NMR (CD₃OD): δ 1.04-1.37 (m, 12H), 1.53 (m, 2H), 1.64-1.76 (m, 6H), 2.38 (m, 2H), 3.03 (s, 12H), 3.46 (m, 4H), 3.85 (m, 4H), 4.52 (s, 4H), 4.70 (s, 4H), 7.24 (m, 2H), 7.34 (m, 12H), 7.43 (s, 2H), 7.52 (m, 6H), 8.20 (d, 2H), 9.02 (d, 2H) ppm.

### ‘Salt Form A’ of [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate

[2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate (as prepared herein above) (107 mg, 0.17 mmol) was dissolved in the minimum quantity of MeCN at RT. The solution was heated and then allowed to cool back to RT. The resulting crystalline solid was filtered off and dried under vacuum. Yield: 83 mg, 78%. Analysis of product prepared by this route was by XRPD identified the product as ‘Salt Form A’.

Preparation [2]

General Experimental Conditions for Preparation [2]

All reactions were carried out under an atmosphere of inert gas unless specified otherwise.

NMR spectra were obtained on a Bruker AVANCE400 spectrometer: Frequency: 400 MHz; 2-Channel; z-Gradient. Temp Range: 0-120° C.

HPLC Conditions:

Phenomenex Luna C18(2) column (50×4.6 mm), 3 μm particle size. UV detection at 210 nm. Elution with A: water+0.05% Trifluoroacetic acid; B: acetonitrile+0.05% Trifluoroacetic acid. Gradient:

| Gradient - Time | flow mL/min | % A | % B |
|---|---|---|---|
| 0.00 | 1.0 | 90 | 10 |
| 8.00 | 1.0 | 10 | 90 |
| 9.00 | 1.0 | 10 | 90 |
| 9.50 | 1.0 | 90 | 10 |
| 12.00 | 1.0 | 90 | 10 |

LC-MS Method: LC-Method as given above. MS: HP-1100 MSD. Detection—API-ES, positive mode.

Preparation [2]

A mixture of (R)-cyclohexyl-(5-dimethylaminomethyloxazol-2-yl)-phenyl-methanol (1 eq.) and 1-(2-bromo-ethoxymethyl)-4-chloro-benzene (2 eq) in 2-propanol (5 Vol.) was heated at 52° C. for 164 h. HPLC showed a conversion of 98%. The reaction mixture was evaporated to dryness to yield [2-(4-chloro-benzyloxy)-ethyl]-[2-((R)-cyclohexyl-hydroxy-phenyl-methyl)-oxazol-5-ylmethyl]-dimethyl-ammonium bromide. The crude sample of [2-(4-chloro-benzyloxy)-ethyl]-[2-((R)-cyclohexyl-hydroxy-phenyl-methyl)-oxazol-5-ylmethyl]-dimethyl-ammonium bromide was dissolved in dichloromethane (4.98 Vol.) and a solution of 1,5-naphthalene disulfonic acid di-sodium salt (1 eq.) in water (10 Vol.) added at room temperature over a period of 10 min. The mixture was diluted with dichloromethane (4.98 Vol.) and stirred for 1 hour at room temperature. The stirrer was turned off and the emulsion settled before separation. To the organic layer was added a mixture of tert-Butyl Methyl Ether (tBME) (10 Vol.) and 2-propanol (1.6 Vol.) at room temperature over a period of 72 min. The resulting suspension was filtered and the cake rinsed with tBME (2.15 Vol.). Drying (rotary evaporator at a bath temperature of 40-50° C. at 5-10 mbar) gave [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate. The yield obtained by this preparation using 130 g of (R)-cyclohexyl-(5-dimethylaminomethyloxazol-2-yl)-phenyl-methanol was 216 g, 83%. ¹H NMR showed a spectrum corresponding to the hemi-salt (2:1 ratio of cation/anion).

Conversion to ‘Salt Form A’ was achieved by suspending a crude batch of [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate as prepared above in acetonitrile (13.8 Vol.). The suspension was heated to reflux and stirred at reflux for 1 hour. Then the suspension was cooled to 70° C. and stirred at this temperature over night. The suspension was cooled to room temperature and the solid filtered and washed with acetonitrile (1.4 Vol.) and dried (rotary evaporator at a bath temperature of 40-50° C. at 5-10 mbar) to yield ‘Salt Form A’. The yield obtained by this conversion using 216 g of crude [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate as starting material was 203.5 g, 94%.

Preparation [3]

General Experimental Conditions for Preparation [3] are the same as for Preparation [2]

A mixture of (R)-cyclohexyl-(5-dimethylaminomethyloxazol-2-yl)-phenyl-methanol (1 eq.) and 1-(2-bromo-ethoxymethyl)-4-chloro-benzene (2 eq.) in 2-propanol (5 Vol.) was submitted to the following temperature program:

Heat to 70° C. (internal temperature) over 1 hour, stir at 70° C. for 26 hours and then cool to 20° C. over 30 minutes. The conversion is checked by HPLC.

The reaction mixture was evaporated to dryness (rotary evaporator at a bath temperature of 40-50° C. at 10-15 mbar) and the residue dissolved in dichloromethane (8.9 Vol.). To the solution was added a solution of 1,5-naphthalene disulfonic acid di-sodium salt (1 eq.) in water (17.7 Vol.) over at least 10 minutes. The resulting mixture was diluted with dichloromethane (8.9 Vol.) and stirring continued at room temperature for 1 hour. The stirrer was turned off and the emulsion settled before separation. To the organic layer was added, over a period of at least 60 minutes at room temperature, a mixture of tBME (17.7 Vol.) and 2-propanol (2.86 Vol.). The suspension formed was stirred at room temperature for 10 to 60 minutes and then filtered. The filter cake is washed with tBME (2×3.46 Vol.) and dried (rotary evaporator at a bath temperature of 40-50° C. at 5-10 mbar) until a Loss On Drying (LOD)≦2 w/w % is obtained. The material was suspended in (22.9 Vol.) of acetonitrile and the suspension submitted to the following temperature program:

Heat to reflux over a period of at least 30 minutes. Stir at reflux for 60 to 70 minutes, then cool to 70° C. (internal temperature) and stir at 70° C. for 16 to 24 hours and finally cool to 20° C. over 1 hour. The suspension was filtered and the filter cake washed with acetonitrile (4.61 Vol.). The material was dried (rotary evaporator at a bath temperature of 40-50° C. at 5-10 mbar) until a LOD≦1 w/w % is obtained.

The yield obtained by this preparation using 25.0 g of (R)-cyclohexyl-(5-dimethylaminomethyloxazol-2-yl)-phenyl-methanol was 38.7 g, 78%.

The yield obtained by this preparation using 129.9 g of (R)-cyclohexyl-(5-dimethylaminomethyloxazol-2-yl)-phenyl-methanol was 203.6 g, 79%.

HPLC and NMR showed a spectrum corresponding to the hemi-salt (2:1 ratio of cation/anion).

### Solid State Analysis of MA 11 Salt Form A of [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate

The melting temperature of Form A as determined by DSC was found to be 233° C. (onset) (±3° C.). Weight loss observed prior to melting by TGA was very low, (from 0.0%-0.5%). GVS determination gave a weight increase of less than 0.5% (% w/w) at 80% RH (±0.3%).

An XRPD spectrum of ‘MA11 Salt Form A’ is presented in FIG. 5.

‘Salt Form A’ was Micronised in a 50 mm jet mill, with ejector pressure 5 bar and milling pressure 1.5-2 bar, giving (90% yield). Particle size of the micronised material as determined by Malvern Laser Diffraction with dry powder feeder was d(0.1) 0.77 μm: d(0.5), 1.45μ: d(0.9): 2.65 μm. An investigational evaluation of the deaggregation properties of micronised ‘Salt Form A’ showed excellent Fine Particle Fraction (FPF >60%) across a range of relative humidity (0-75% RH).

Biological Activity of Muscarinic Antagonists

The inhibitory effects of compounds of the muscarinic antagonists were determined by a Muscarinic Receptor Radioligand Binding Assay.

Radioligand binding studies utilising [³H]-N-methyl scopolamine ([³H]-NMS) and commercially available cell membranes expressing the human muscarinic receptors (M2 or M3) were used to assess the affinity of muscarinic antagonists for M2 and M3 receptors. Membranes in TRIS buffer were incubated in 96-well plates with [³H]-NMS and M3 antagonist at various concentrations for 3 hours. Membranes and bound radioligand were then harvested by filtration and allowed to dry overnight. Scintillation fluid was then added and the bound radioligand counted using a Canberra Packard Topcount scintillation counter

The half-life of antagonists at each muscarinic receptor was measured using the alternative radioligand [³H]-QNB and an adaptation of the above affinity assay. Antagonists were incubated for 3 hours at a concentration 10-fold higher than their Ki, as determined with the [³H]-QNB ligand, with membranes expressing the human muscarinic receptors. At the end of this time, [³H]-QNB was added to a concentration 25-fold higher than its Kd for the receptor being studied and the incubation continued for various time periods from 15 minutes up to 180 minutes. Membranes and bound radioligand were then harvested by filtration and allowed to dry overnight. Scintillation fluid was then added and the bound radioligand counted using a Canberra Packard Topcount scintillation counter.

The rate at which [³H]-QNB is detected binding to the muscarinic receptors is related to the rate at which the antagonist dissociates from the receptor, i.e. to the half life of the antagonists on the receptors.

The following compounds were tested in the receptor binding assay:

| Muscarinic | M3 binding |
|---|---|
| Antagonist | Ki, nM |
| MA 1 | 9.4 |
| MA 2 | 0.2 |
| MA 3 | 0.6 |
| MA 4 | 0.9 |
| MA 5 | 2.1 |
| MA 6 | 0.6 |

Preparation of β₂-adrenoceptor Agonists

The following β₂-adrenoceptor agonists that may be employed in the combination of the present invention may be prepared as follows.

General Experimental Details for Preparation of β₂-adrenoceptor Agonists

¹H NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 8 min, 95% B 1 min.

Analytical chromatography was run on a Symmetry C₁₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

The abbreviations or terms used in the examples have the following meanings:

SCX: Solid phase extraction with a sulfonic acid sorbent

HPLC: High performance liquid chromatography

DMF: N,N-Dimethylformamide

The β₂-adrenoceptor agonists and the intermediates used in their preparation are herein named, based upon the structures depicted, using the IUPAC NAME, ACD Labs Version 8 naming package.

### β₂-Adrenoceptor Agonist 1: (BA1): Preparation 1

### N-[2-(Diethylamino)ethyl]-N-(2-{[1-(4-hydroxy-2-oxo-2,3-dihydro-1,3-benzothiazol-7-yl)ethyl]amino}ethyl)-3-[1-(1-naphthyl)ethoxy]propanamide dihydrobromide

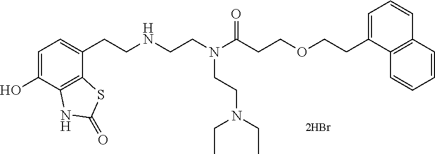

### a) tert-Butyl 3-[2-(1-naphthyl)ethoxy]propanoate

1-Naphthalene ethanol (10 g) was treated with benzyltrimethylammonium hydroxide (Triton B®; 0.9 mL of a 40% solution in methanol) and the resulting mixture stirred in vacuo for 30 minutes. The mixture was then cooled to 0° C. and treated with tert-butyl acrylate (8.19 g). The resulting mixture was slowly warmed to room temperature and stirred overnight. The crude mixture was subsequently absorbed onto aluminium oxide (30 g) and eluted with diethylether (200 mL). The organics were concentrated to give a crude material (16.6 g) which was purified by flash silica chromatography eluting with 1:8, diethylether: hexane to give the subtitled compound (12.83 g).

¹H NMR (CDCl₃) δ 8.05 (dd, 1H), 7.84 (dd, 1H), 7.72 (dd, 1H), 7.54-7.34 (m, 4H), 3.81-3.69 (m, 4H), 3.35 (t, 2H), 2.52-2.47 (m, 2H), 1.45 (s, 9H).

### b) 3-[2-(1-Naphthyl)ethoxy]propanoic acid

tert-Butyl 3-[2-(1-naphthyl)ethoxy]propanoate (6.19 g) was taken up in dichloromethane (30 mL) and treated with trifluoroacetic acid (5 mL). The resulting solution was stirred at room temperature for 2 hours, an additional 1 mL of trifluoroacetic acid was added and the solution stirred overnight. The mixture was concentrated, taken up in 2M sodium hydroxide solution (30 mL) and washed with ether (2×20 mL). The aqueous layer was subsequently acidified (using 1M hydrochloric acid) and extracted with ether (2×30 mL). The combined organics were washed with brine (20 mL), dried over anhydrous magnesium sulphate, filtered and concentrated in vacuo to give the sub-titled compound (5.66 g) as a clear oil.

¹H NMR (CDCl₃) δ 8.05 (bs, 1H), 7.85 (bs, 1H), 7.74 (bs, 1H), 7.50-7.38 (m, 4H), 3.84-3.75 (bm, 4H), 3.39 (bs, 2H), 2.65 (bs, 2H).

### c) N-(2-Diethylaminoethyl)-N-(2-hydroxyethyl)-3-[2-(1-naphthyl)ethoxy]-propanamide

Oxalyl chloride (0.33 g) was added dropwise to a solution of 3-[2-(1-naphthyl)ethoxy]propanoic acid (0.53 g) in dichloromethane (10 mL), dimethylformamide (1 drop) was added and stirring continued at room temperature for 1 hour. The mixture was subsequently concentrated, re-dissolved in dichloromethane (10 mL) and added dropwise to a solution of 2-(2-diethylaminoethylamino)ethanol (0.35 g) and diisopropylethylamine (0.56 g) in dichloromethane (10 mL). The resulting mixture was stirred at room temperature for 1 hour, diluted (dichloromethane, 50 mL), washed with water (2×20 mL), brine (20 mL), dried over magnesium sulfate and concentrated to give the crude product (0.91 g) which was purified by flash column chromatography (eluting with 5-7% methanol in dichloromethane) to give 0.63 g of the sub-titled compound.

¹H NMR (CDCl₃) δ 8.05 (d, 1H), 7.85 (d, 1H), 7.73 (d, 1H), 7.52-7.47 (m, 2H), 7.42-7.35 (m, 2H), 3.84-3.78 (m, 6H), 3.72-3.70 (m, ½H), 3.45-3.35 (m, 6H), 2.79-2.77 (m, 1+½H), 2.62-2.58 (m, 2H), 2.54-2.49 (m, 4H), 1.04-1.01 (m, 6H).

### d) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide

A solution of dimethylsulfoxide (0.097 g) in dichloromethane (1 mL) was added to a solution of oxalyl chloride (0.079 g) in dichloromethane (10 mL) at −78° C. The reaction was stirred for 15 minutes and then a solution of N-(2-diethylaminoethyl)-N-(2-hydroxyethyl)-3-[2-(1-naphthyl)ethoxy]propanamide (0.22 g) in dichloromethane (1 mL+1 mL wash) was added and the reaction mixture stirred for a further 15 minutes. Triethylamine (0.29 g) was added and the reaction allowed to warm to room temperature over 1 hour, the mixture was subsequently diluted (dichloromethane 30 mL), the organics washed with sodium bicarbonate (20 mL), brine (20 mL), dried over anhydrous magnesium sulphate, filtered and concentrated in vacuo to give the sub-titled compound (0.21 g).

The crude product was dissolved in methanol (10 mL) and 7-(2-aminoethyl)-4-hydroxy-1,3-benthiazol-2(3H)-one hydrochloride (prepared according to the procedure outlined in Organic Process Research & Development 2004, 8(4), 628-642; 0.131 g) was added along with acetic acid (0.1 mL) and water (0.1 mL). After stirring at room temperature for 30 minutes, sodium cyanoborohydride (0.020 g) was added and the reaction mixture was stirred overnight. Ammonia (7N in methanol, 1 mL) was added and the mixture was concentrated. The crude residue was purified by flash column chromatography eluting with 1% ammonia; 5%-7% methanol in dichloromethane. The crude product was used directly in the next step.

### e) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

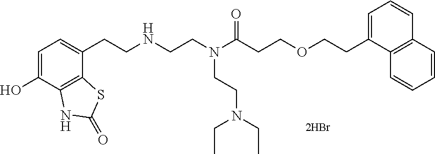

N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide (0.052 g) was dissolved in ethanol (1.5 mL) and treated with 48% hydrobromic acid (21 μl). The white solid dihydrobromide salt (0.058 g) was collected by filtration.

MS: APCI (+ve) 579 (M+1)

¹H NMR δ(DMSO) 11.78-11.71 (m, 1H), 10.11-10.06 (m, 1H), 9.51-9.43 (m, 0.33H), 9.21-9.13 (m, 0.66H), 8.75-8.66 (m, 1H), 8.59-8.51 (m, 1H), 8.06 (d, 1H), 7.95-7.90 (m, 1H), 7.79 (d, 1H), 7.60-7.48 (m, 2H), 7.47-7.39 (m, 2H), 6.87 (t, 1H), 6.76 (dd, 1H), 3.78-3.53 (m, 10H), 3.25-3.09 (m, 10H), 2.91-2.80 (m, 2H), 2.73-2.61 (m, 2H), 1.26-1.15 (m, 6H). NMR indicates approximately 2:1 mixture of rotamers at 298K.

### δ₂-Adrenoceptor Agonist 1: (BA1): Preparation 2

### N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

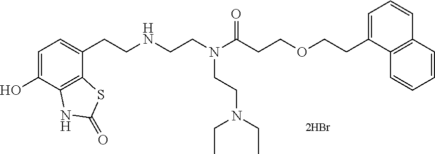

### a) N′-(2,2-Dimethoxyethyl)-N,N-diethyl-ethane-1,2-diamine

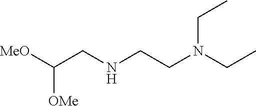

A solution of N,N-diethyl-ethylenediamine (150 g) in methanol (500 mL) was treated dropwise rapidly with glyoxal dimethylacetal (60 wt % soln. in water, 225 g) at 10-15° C. After the addition was complete the solution was warmed to 15° C., then to 22° C. and left at this temperature for 16 hours. The reaction mixture was treated with 5% palladium on carbon (Johnson-Matthey type 38H paste, 15 g) and hydrogenated at 6 bar until the reaction was complete as judged by GC/MS. The catalyst was removed by filtration and the filtrate evaporated to dryness (toluene azeotrope, 2.5 L), affording 196.2 g of the sub-titled compound.

¹H NMR (CDCl₃): 4.48 (t, 1H), 3.39 (s, 6H), 2.75 (d, 2H), 2.69 (t, 2H), 2.57-2.48 (m, 6H), 1.01 (ts, 6H).

### b) N-[2-(Diethylamino)ethyl]-N-(2,2-dimethoxyethyl)-3-[2-(1-naphthyl)ethoxy]propanamide

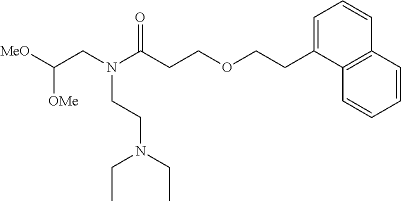

Oxalyl chloride (151 mL) was added dropwise over 45 minutes to a solution of 3-[2-(1-naphthyl)ethoxy]propanoic acid (389 g) (Example 7 step b)) in dichloromethane (2.1 L) and DMF (0.5 mL). The reaction mixture was stirred for a further 16 hours. The mixture was subsequently concentrated, redissolved in DCM (1.7 L) and added dropwise over 1.75 hours at 0° C. to a solution of N′-(2,2-dimethoxyethyl)-N,N-diethylethane-1,2-diamine (325 g) and isopropyldiethylamine (551 mL) in DCM (1.7 L). The resulting mixture was stirred at room temperature for 3 hours, washed with aqueous saturated sodium bicarbonate solution (5×1 L), water (1.5 L) and dried over sodium sulphate and concentrated to give 650 g of the sub-titled compound.

m/e 431 (M+H⁺, 100%)

### c) N-[2-(Diethylamino)ethyl]-3-[2-(1-naphthyl)ethoxy]-N-(2-oxoethyl)propanamide

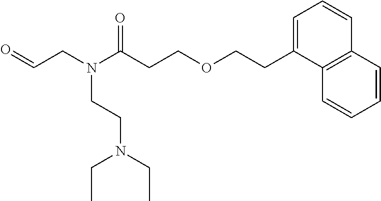

A solution of N-[2-(diethylamino)ethyl]-N-(2,2-dimethoxyethyl)-3-[2-(1-naphthyl)ethoxy]propanamide (93 g) in DCM (270 mL) was treated dropwise at 0° C. with trifluoroacetic acid (270 mL) over 1.5 hours. After the addition the reaction mixture was allowed to warm to room temperature and stirred for a further 1 hour. The reaction mixture was concentrated and the residue poured into aqueous saturated sodium bicarbonate solution (1800 mL, caution). The aqueous mixture was extracted with DCM (4×400 mL) and the combined extracts were dried over magnesium sulphate and concentrated. The residue was used directly in the following reaction.

### d) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

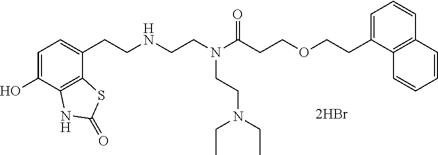

A suspension of 7-(2-amino-ethyl)-4-hydroxy-3H-benzothiazol-2-one hydrochloride (53 g) in dry NMP (216 mL) was heated to 60° C. and treated in one portion with a solution of NaOH (8.2 g) in methanol (102 mL). The bright orange suspension was cooled to room temperature and treated dropwise with a solution of N-[2-(diethylamino)ethyl]-3-[2-(1-naphthyl)ethoxy]-N-(2-oxoethyl)propanamide in dichloromethane (475 mL) over 20 minutes. The reaction was left to stir for 25 minutes. Sodium triacetoxyborohydride (91.5 g) was then added in portions over 20 minutes and the mixture stirred for a further 50 minutes. The reaction mixture was poured into water (1.8 L) and the acidic solution (pH5) was washed with tea butyl methyl ether (TBME) (3×500 mL). The aqueous phase was basified to pH8 by the addition of solid potassium carbonate and extracted with dichloromethane (3×750 mL); the combined organic extracts were dried over magnesium sulphate and concentrated to give a dark oil. This was dissolved in ethanol (200 mL) and 48% aqueous hydrobromic acid (73 mL) was added. The solution was aged for 30 minutes then evaporated to dryness. The residue was triturated with ethanol (560 mL); the resultant solid was collected by filtration and dried in vacuo at 50° C. The sticky solid was suspended in boiling ethanol (100 mL) and filtered while hot. The collected solid was dried in vacuo at 50° C. This material was recrystallised from ethanol/water (3:1, 500 mL). After standing overnight the resultant solid was collected by filtration and washed with ice-cold ethanol (75 mL). Drying in vacuo at 50° C. for 24 hr afforded 57 g of the title compound.

### β₂-Adrenoceptor Agonist 2: (BA2)

### N-[2-(Diethylamino)ethyl]-N-(2-{[2-(4-hydroxy-2-oxo-2,3-dihydro-1,3-benzothiazol-7-yl)ethyl]amino}-3-[2-(3-chlorophenyl)ethoxy]propanamide dihydrobromide

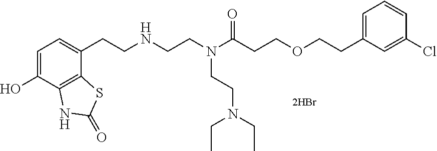

### a) tert-Butyl 3-[2-(3-chlorophenyl)ethoxy]propanoate

2-(3-chlorophenyl)ethanol (20 g) was treated with benzyltrimethylammonium hydroxide (Triton B®) (2.67 mL) and the resultant mixture was stirred in vacuo for 30 minutes. The mixture was then cooled to 0° C. and treated with t-butyl acrylate (17.40 g). The reaction was warmed to room temperature and stirred for 16 hours. The mixture was filtered through aluminium oxide (15 g) eluting with ether (75 mL). The collected filtrate was concentrated to give the sub-titled compound (34.40 g) as an oil.

¹H NMR (CDCl₃) [symbol] 7.26-7.07 (m, 4H), 3.69-3.59 (m, 4H), 2.86-2.81 (t, 2H), 2.50-2.45 (t, 2H), 1.43 (s, 9H)

### b) 3-[2-(3-chlorophenyl)ethoxy]propanoic acid

tert-Butyl 3-[2-(3-chlorophenyl)ethoxy]propanoate (example 1a), 34.40 g) was dissolved in dichloromethane (150 mL) and treated with trifluoroacetic acid (50 mL). The mixture was stirred at room temperature for 3 hours, then concentrated in vacuo and azeotroped with dichloromethane (2×10 mL). The residue was taken up in dichlormethane (300 mL) and extracted with saturated sodium hydrogen carbonate (200 mL). The basic layer was washed with dichloromethane (20 mL) then acidified with 2M hydrochloric acid. The acidic layer was extracted with dichloromethane (2×200 mL). The organic layers were combined, washed with brine, dried over anhydrous magnesium sulphate, filtered and concentrated to yield the sub-titled compound (24.50 g) as an oil.

m/e 227 [M−H]

### c) N-[2-(Diethylamino)ethyl]-N-(2,2-dimethoxyethyl)-3-[2-(3-chlorophenyl)ethoxy]propanamide

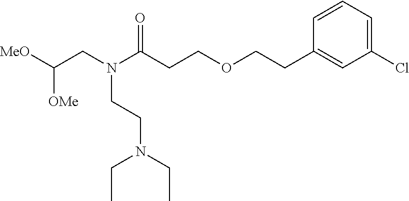

Oxalyl chloride (9.50 mL) was added dropwise over 45 minutes to a solution of 3-[2-(3-chlrophenyl)ethoxy]propanoic acid (22.50 g) (example 1b) in dichloromethane (120 ml) and DMF (0.5 mL). The reaction mixture was stirred for a further 16 hours. The mixture was subsequently concentrated, redissolved in DCM (1.7 L) and added dropwise over 1.75 hours at 0° C. to a solution of N′-(2,2-dimethoxyethyl)-N,N-diethylethane-1,2-diamine (20.20 g) (example 16a) and isopropyldiethylamine (34.43 mL) in DCM (200 mL). The resulting mixture was stirred at room temperature for 16 hours, washed with aqueous saturated sodium bicarbonate solution (3×1 L), water (1.5 L) and dried over sodium sulphate and concentrated to give 39.50 g of the sub-titled compound.

m/e 415 (M+H⁺, 83%)

### d) N-[2-(Diethylamino)ethyl]-3-[2-(3-chlorophenyl)ethoxy]-N-(2-oxoethyl)propanamide

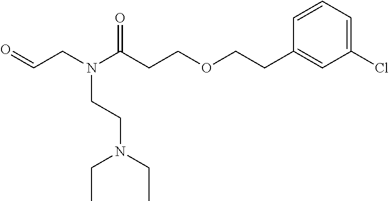

A solution of N-[2-(Diethylamino)ethyl]-N-(2,2-dimethoxyethyl)-3-[2-(3-chlorophenyl)ethoxy]propanamide (example 1c) (20 g) in DCM (500 mL) was treated dropwise at 0° C. with trifluoroacetic acid (50 mL) over 30 minutes. After the addition the reaction mixture was allowed to warm to room temperature and stirred for a further 1 hour. The reaction mixture was concentrated and the residue poured into aqueous saturated sodium bicarbonate solution (1800 mL, caution). The aqueous mixture was extracted with DCM (3×400 mL) and the combined extracts were dried over magnesium sulphate and concentrated. The residue was used directly in the following reaction.

### e) N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide

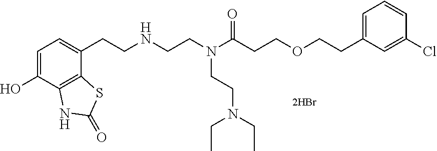

A suspension of 7-(2-amino-ethyl)-4-hydroxy-3H-benzothiazol-2-one hydrochloride (11.77 g) in dry NMP (50 mL) was heated to 65° C. and treated in one portion with a solution of NaOH (1.83 g) in methanol (23 mL). The bright orange suspension was cooled to room temperature and treated dropwise with a solution of N-[2-(diethylamino)ethyl]-3-[2-(3-chlorophenyl)ethoxy]-N-(2-oxoethyl)propanamide (example 1d) in dichloromethane (50 mL) over 30 minutes. The reaction was left to stir for 30 minutes. Sodium triacetoxyborohydride (20.33 g) was then added in portions over 20 minutes and the mixture stirred for a further 16 hours. The reaction mixture was poured into water (1.8 L), basified to pH8 by the addition of solid potassium carbonate and extracted with dichloromethane (2×500 mL); the combined organic extracts were dried over magnesium sulphate and concentrated to give a dark oil. The residue was purified by chromatography on silica with 10% (0.1% aqNH₃/MeOH)/DCM as eluent to give the sub-title compound as a brown oil. Yield (6.58 g). This was dissolved in ethanol (150 mL) and 48% aqueous hydrobromic acid (10 mL) was added. The solution was aged for 30 minutes then evaporated to dryness. The residue was triturated with ethanol (100 mL); the resultant solid was collected by filtration and dried in vacuo at 50. This material was recrystallised from ethanol/water (6:1, 500 mL); after standing overnight the resultant solid was collected by filtration and washed with ice-cold ethanol (75 mL). Drying in vacuo at 50° C. for 24 hr afforded 4.96 g of the title compound.

MS: APCI (+ve): 563 (M+1) 99.3% purity (T9505M).

¹H NMR (DMSO, 90° C.), δ 11.75-11.73 (m, 1H), 10.08-10.06 (d, 1H), 8.65 (bs, 1H), 7.33-7.19 (m, 4H), 6.89-6.84 (t, 1H), 6.77-6.74 (m, 1H), 3.68-3.58 (m, 8H), 3.17-3.16 (m, 10H), 2.86-2.80 (m, 4H), 2.67-2.62 (m, 2H), 1.23-1.19 (t, 6H).

Elemental Analysis

CHNS C, 46.54% (46.39); H, 5.75% (5.70); N, 7.94% (7.73); S, 4.46% (4.42).

### β₂-Adrenoceptor Agonist 3: (BA3)

### 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide

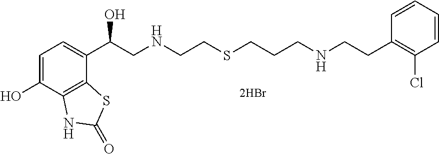

### a) 1-Chloro-2-[(E)-2-nitrovinyl]benzene

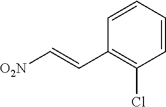

2-Chlorobenzaldehyde (ex Aldrich) (10.0 g) was mixed with nitromethane (26.05 g) and ammonium acetate (21.92 g) in acetic acid (200 mL), and the mixture was heated at reflux for 40 minutes. The mixture was allowed to cool to room temperature, and the majority of the acetic acid was removed in vacuo. The residue was dissolved in dichloromethane and washed with water, then potassium carbonate solution (×2), then water again. The organics were dried over anhydrous magnesium sulfate, filtered and evaporated to give the desired material, as an orange oil (12.83 g).

¹H NMR δ(CDCl₃) 8.41 (d, 1H), 7.62-7.57 (m, 2H), 7.52-7.48 (m, 1H), 7.43 (dt, 1H), 7.34 (ddd, 1H)

### b) 2-(2-Chlorophenyl)ethanamine

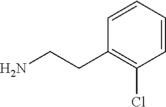

Aluminium hydride was prepared by the drop-wise addition of a solution of sulphuric acid (8.40 mL) in dry THF (60 mL) to a stirred solution of 1.0M lithium aluminium hydride in THF (314 mL), at 0-10° C., under a nitrogen atmosphere. After stirring at 5° C. for 30 minutes, a solution of 1-chloro-2-[(E)-2-nitrovinyl]benzene (12.83 g) in dry THF (160 mL) was added dropwise maintaining the internal temperature between 0° C. and 10° C. When the addition was complete the reaction was heated at reflux for 5 minutes. The mixture was allowed to cool to room temperature, then cooled to 0° C. and isopropanol (22 mL) carefully added dropwise maintaining the temperature below 20° C. 2M Sodium hydroxide (35 mL) was carefully added dropwise maintaining the temperature below 20° C. The mixture was stirred at room temperature for 30 minutes, then filtered through a layer of celite, which was then washed with THF (×3). The filtrate was evaporated to dryness. The residue was purified using silica column chromatography, using ethyl acetate to load the material, then 10% triethylamine in ethyl acetate, followed by 10% triethylamine in 45% ethanol: 45% ethyl acetate as the eluents, to give the desired material (4.66 g).

¹H NMR δ(CDCl₃) 7.36 (dd, 1H), 7.25-7.13 (m, 3H), 2.98 (dt, 2H), 2.91-2.87 (m, 2H)

### c) tert-Butyl [2-(2-chlorophenyl)ethyl]carbamate

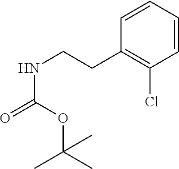

To a stirred solution of 2-(2-chlorophenyl)ethanamine (25.57 g) and triethylamine (22.87 mL) in dry THF (300 mL) was added a solution of di-tert-butyl dicarbonate (35.85 g) in dry THF (50 mL) over 10 minutes, at ambient temperature, under a nitrogen atmosphere. The reaction mixture was stirred at room temperature for 3 hours. The solvents were removed in vacuo to give the desired material, as a yellow oil (42.0 g).

¹H NMR δ(CDCL3) 7.35 (d, 1H), 7.25-7.14 (m, 3H), 4.57 (s, 1H), 3.43-3.35 (m, 2H), 2.95 (t, 2H), 1.43 (d, 9H)

### d) tert-Butyl allyl[2-(2-chlorophenyl)ethyl]carbamate

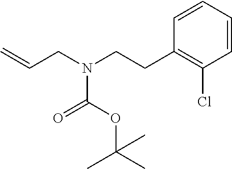

To a suspension of sodium hydride (60% in mineral oil) (7.23 g), which had been washed with ether (×3), in dry DMF (200 mL) was added a solution of tert-butyl [2-(2-chlorophenyl)ethyl]carbamate (42.0 g) in dry DMF (50 mL), over a 15 minute period, at 35° C., under a nitrogen atmosphere. When the addition was complete, the mixture was stirred at 50° C. for 90 minutes. The mixture was allowed to cool to room temperature, then allyl bromide (15.63 mL) was added slowly, keeping the temperature at 25° C., using external cooling. The mixture was stirred at room temperature for 2 hours, then diluted with water and extracted with ethyl acetate (×3). The organics were combined, washed with water, dried over anhydrous magnesium sulfate, filtered and evaporated. The residue was purified using silica column chromatography, loading with 1% ethyl acetate in isohexane, then using isohexane with ethyl acetate (0%, 1%, 2%, %5) as the eluents to give the desired material (27.0 g). There were several mixed fractions, so these were combined, and re-purified using silica column chromatography, as above, to give a further 4 g of desired material. Both crops of product were combined to give 31.0 g in total.

¹H NMR δ(CDCl₃) 7.36-7.31 (m, 1H), 7.21-7.12 (m, 3H), 5.83-5.68 (m, 1H), 5.17-5.05 (m, 2H), 3.86-3.66 (m, 2H), 3.41 (t, 2H), 3.03-2.90 (m, 2H), 1.43 (s, 9H)

HPLC: 95.90% @ 220 nm [M+H-Boc]+=196.1 (Calc=295.1339) (multimode+)

### e) tert-Butyl [2-(2-chlorophenyl)ethyl]{3-[(2-hydroxyethyl)thio]propyl}carbamate

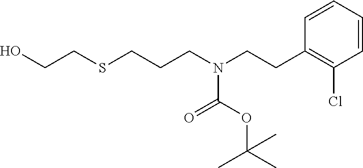

tert-Butyl allyl[2-(2-chlorophenyl)ethyl]carbamate (31.0 g) was mixed with 2-mercaptoethanol (7.37 mL), and AIBN (1.15 g), and stirred at 65° C. for 45 minutes. The mixture was cooled and more mercaptoethanol (1 mL) and AIBN (200 mg) added. The mixture was then heated at 65° C. for a further 30 minutes. The material was purified by silica column chromatography, loading the material in 20% ethyl acetate in isohexane, then eluting with 20% ethyl acetate in isohexane, changing to 50%, to give the desired material (31.94 g).

¹H NMR δ(CDCl₃) 7.38-7.32 (m, 1H), 7.22-7.13 (m, 3H), 3.75-3.68 (m, 2H), 3.41 (t, 2H), 3.32-3.14 (m, 2H), 3.03-2.91 (m, 2H), 2.72 (t, 2H), 2.54-2.36 (m, 2H), 1.85-1.71 (m, 2H), 1.42 (s, 9H)

HPLC: 92.31% @ 220 nm [M+H-Boc]+=274.1 (Calc=373.1478) (multimode+)

### f) tert-Butyl [2-(2-chlorophenyl)ethyl]{3-[(2-oxoethyl)thio]propyl}carbamate

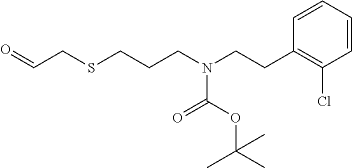

Sulfur trioxide:pyridine complex (30.52 g) was dissolved in DMSO (200 mL) and stirred at room temperature, under a nitrogen atmosphere, for 15 minutes. DCM (100 mL) was added, followed by a solution of tert-butyl [2-(2-chlorophenyl)ethyl]{3-[(2-hydroxyethyl)thio]propyl}carbamate (23.9 g) and Hunigs base (63.5 mL) in DCM (160 mL), which was added in one portion (exotherm). The resulting mixture was stirred at ambient temperature for 15 minutes. The reaction mixture was diluted with ethyl acetate, washed with water, then 1N HCl, then saturated sodium bicarbonate solution, dried over anhydrous magnesium sulfate, filtered and the solvents removed in vacuo. The material was purified by silica column chromatography eluting with 20% ethyl acetate in isohexane to give the desired material (12.43 g).

¹H NMR δ(CDCl₃) 9.46 (t, 1H), 7.36-7.32 (m, 1H), 7.21-7.13 (m, 3H), 3.40 (t, 2H), 3.29-3.13 (m, 4H), 3.02-2.90 (m, 2H), 2.45-2.34 (m, 2H), 1.82-1.69 (m, 2H), 1.49-1.36 (m, 9H)

### g) tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

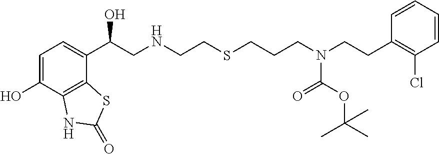

The tert-butyl [2-(2-chlorophenyl)ethyl]{3-[(2-oxoethyl)thio]propyl}carbamate (11.32 g) was dissolved in a mixture of methanol (200 mL) and acetic acid (1.74 ml). 7-[(1R)-2-amino-1-hydroxyethyl]-4-hydroxy-1,3-benzothiazol-2(3H)-one hydrochloride (8.0 g) was added to the solution, and the mixture stirred at room temperature, under a nitrogen atmosphere, for 1 hour. Sodium cyanoborohydride (1.92 g) was added and the mixture stirred for a further 2 hours. The solvents were removed in vacuo, and the residue diluted with water, basified with 0.880 aqueous ammonia, and extracted with ethyl acetate (×3) (filtered through celite during extraction). The organics were combined, washed with brine, dried over anhydrous sodium sulfate, filtered and evaporated to give a brown residue (15.5 g). The material was purified using silica column chromatography, using DCM with MeOH (2%, 5%, 10%, 20% and 30%, all with 1% 0.880 aq NH₃) as the eluent, to give the desired material (6.67 g) (38% yield)

¹H NMR δ(DMSO) 7.43-7.38 (m, 1H), 7.30-7.21 (m, 3H), 6.86 (d, 1H), 6.69 (d, 1H), 4.56 (dd, 1H), 3.23-3.10 (m, 2H), 2.88 (t, 2H), 2.71-2.48 (m, 8H), 2.46-2.39 (m, 2H), 1.72-1.62 (m, 2H), 1.40-1.22 (m, 9H)

HPLC: 97.46% @ 220 nm [M+H]+=582.1 (Calc=582.1863) (multimode+)

### h) 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide

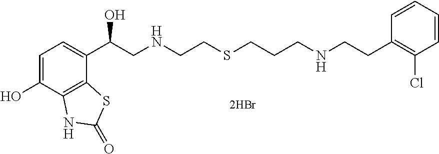

To a stirred suspension of the Boc compound from part g) (5.93 g) in DCM (20 mL) was added trifluoroacetic acid (20 mL) at 0° C., and the resulting mixture was stirred under nitrogen for 30 minutes. The mixture was diluted with toluene, and solvents removed, then azeotroped with toluene (×2). The residue was dissolved in acetonitrile, acidified with 48% aq HBr and concentrated in vacuo (not to dryness). The mixture was further diluted with acetonitrile and the precipitated solid collected by filtration, washed with acetonitrile and dried under vacuum to give 6.35 g. A 3.8% impurity was present (isomer from part e)), so the material was redissolved in a 1:1 mixture of acetonitrile:water and purified using prep HPLC (Sunfire 30×80 mm C8 column; NH₄OAc buffer; acetonitrile 5-50% over 10 minutes). The resultant material was dried overnight in a dessicator at 10 mbar over KOH and H₂SO₄. The resulting di-acetate salt was dissolved in water and basified with 0.880 aq ammonia. A white gum formed, so the aqueous was decanted off, and the gum dried in vacuo to give the free base (4.11 g). This was dissolved in hot ethanol, and the solution was filtered, then allowed to cool to room temperature. The solution was acidified with 48% aq. HBr and left to crystallize. The white solid was collected by filtration, washed with ethanol and dried in vacuo to give 3.81 g Crop 1.

¹H NMR δ(DMSO) 11.67 (s, 1H), 10.15 (s, 1H), 8.70 (s, 4H), 7.50-7.30 (m, 4H), 6.94 (d, 1H), 6.78 (d, 1H), 6.45 (s, 1H), 4.96-4.90 (m, 1H), 3.22-3.02 (m, 10H), 2.86-2.76 (m, 2H), 2.66 (t, 2H), 1.91 (quintet, 2H)

HPLC: 99.63% @ 220 nm [M+H]+=482 (calc=482.1339) (MultiMode+)

| | | Elemental analysis: | | | |
|---|---|---|---|---|---|
| | | C | H | N | S |
| Calculated: | | 41.04 | 4.70 | 6.53 | 9.96 |
| Found: | 1: | 41.07 | 4.69 | 6.67 | 9.72 |
| | 2: | 41.08 | 4.68 | 6.74 | 9.67 |
| | 3: | 40.96 | 4.68 | 6.75 | 9.67 |

The mother liquors were evaporated to dryness then triturated with acetonitrile. The solid was collected by filtration to give 719 mg Crop 2 (4.53 g total).

¹H NMR δ(DMSO) 11.67 (s, 1H), 10.15 (s, 1H), 8.80-8.60 (m, 4H), 7.50-7.29 (m, 4H), 6.94 (d, 1H), 6.78 (d, 1H), 6.45 (s, 1H), 4.96-4.89 (m, 1H), 3.22-3.00 (m, 10H), 2.85-2.76 (m, 2H), 2.66 (t, 2H), 1.90 (quintet, 2H)

HPLC: 99.20% @ 220 nm [M+H]+=482 (calc=482.1339) (MultiMode+)

| | | Elemental analysis: | | | |
|---|---|---|---|---|---|
| | | C | H | N | S |
| Calculated: | | 41.04 | 4.70 | 6.53 | 9.96 |
| Found: | 1: | 40.90 | 4.69 | 6.78 | 9.60 |
| | 2: | 41.01 | 4.70 | 6.83 | 9.60 |
| | 3: | 40.97 | 4.69 | 6.76 | 9.63 |

Biological Activity of β₂-Adrenoceptor Agonist

Adrenergic β2 Mediated cAMP Production

Cell Preparation

H292 cells were grown in 225 cm2 flasks incubator at 37° C., 5% CO₂ in RPMI medium containing, 10% (v/v) FBS (foetal bovine serum) and 2 mM L-glutamine.

Experimental Method

Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time Compound A was added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. A concentration response curve for Compound A was constructed and data was fitted to a four parameter logistic equation to determine both the pEC₅₀ and Intrinsic Activity. Intrinsic Activity was expressed as a fraction relative to the maximum activity determined for formoterol in each experiment. Result are in Table 1.

Selectivity Assays

Adrenergic

Membrane Preparation

Membranes were prepared from human embryonic kidney 293 (HEK293) cells expressing recombinant human α1_{D} receptor. These were diluted in Assay Buffer (50 mM HEPES, 1 mM EDTA, 0.1% gelatin, pH 7.4) to provide a final concentration of membranes that gave a clear window between maximum and minimum specific binding.

Experimental Method

Assays were performed in U-bottomed 96-well polypropylene plates. 10 μL [³H]-prazosin (0.3 nM final concentration) and 10 μL of Compound A (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScierice) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.1 nM to 10 μM. Data was fitted to a four parameter logistic equation to determine the compound potency, which was expressed as pIC50 (negative log molar concentration inducing 50% inhibition of [³H]-prazosin binding). Results are shown in Table 1 below.

Adrenergic β1

Membrane Preparation

Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

Experimental Method

Assays were performed in U-bottomed 96-well polypropylene plates. 10 μL [¹²⁵I]-Iodocyanopindolol (0.036 nM final concentration) and 10 μL of Compound A (10× final concentration) were added to each test well. For each assay plate 8 replicates were obtained for [¹²⁵I]-Iodocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated ORB filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [¹²⁵I]-Iodocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-Iodocyanopindolol binding). Results are shown in Table 1 below.

Dopamine D2

Membrane Preparation

Membranes containing recombinant human Dopamine Subtype D2s receptors were obtained from Perkin Elmer. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

Experimental Method

Assays were performed in U-bottomed 96-well polypropylene plates. 30 μL [³H]-spiperone (0.16 nM final concentration) and 30 μL of Compound A (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-spiperone binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [³H]-spiperone binding). Results are shown in Table 1.

TABLE 1

| | | | | β1 bind | D2 bind |
|---|---|---|---|---|---|
| Compound | β2 pEC50 | β2 Int Act | α1 bind pIC50 | p IC50 | pIC50 |
| BA1 | 8.2 | 0.8 | 6.6 | <5 | 6.1 |
| BA2 | 8.3 | 0.7 | <6.1 | <5 | 5.6 |
| BA3 | 9.2 | 0.8 | 7.6 | 6.9 | 5.8 |

In Vitro Combination Data

Evaluation of Compound Activity on Isolated Tracheal Rings from Guinea-Pig Preconstricted with Methacholine.

Addition of β2-adrenoceptor agonists and/or muscarinic M3 receptor antagonists causes relaxation of isolated guinea-pig tracheal rings precontracted with the muscarinic agonist, methacholine. Male albino Dunkin Hartley guinea-pigs (300-350 g) were killed by cervical dislocation and the trachea excised. Adherent connective tissue was removed and the trachea cut into ring segments (2-3 mm wide). These were suspended in 10 mL organ baths containing a modified Krebs solution composition (mM): NaCl 117.56, KCl 5.36, NaH₂PO₄ 1.15, MgSO₄ 1.18, glucose 11.10, NaHCO₃ 25.00 and CaCl₂ 2.55. This was maintained at 37° C. and continually gassed with 5% CO₂ in O₂, Indomethacin (2.8 μM), corticosterone (10 μM), ascorbate (1 mM), CGP20712A (1 μM) and phentolamine (3 μM) were added to the Krebs solution: indomethacin to prevent development of smooth muscle tone due to the synthesis of cyclooxygenase products, corticosterone to inhibit the uptake 2 process, ascorbate to prevent catecholamine oxidation and CGP20712A and phentolamine to avoid any complicating effects of β1- and α-adrenoceptor activation respectively. The tracheal rings were suspended between two stainless steel hooks, one attached to an isometric force transducer and the other to a stationary support in the organ bath. Changes in isometric force were recorded. Acetyl-β-methylcholine chloride (Methacholine), Indomethacin, Corticosterone-21-acetate, Phentolamine hydrochloride, Ascorbic acid, CGP20712A methanesulphate were obtained from the Sigma Chemical Company. Indomethacin was dissolved in 10% w/v Na₂CO₃, corticosterone 21-acetate in ethanol and other compounds in DMSO. Muscarinic Antagonists (MA2), (MA11) and formoterol were diluted in Krebs prior to adding to tissues and the level of DMSO in the bath was <0.1%.

### Muscarinic Antagonist 2 (MA2): [2-((R)-Cyclohexyl-hydroxy-phenyl-methyl)-oxazol-5-ylmethyl]-dimethyl-(3-phenoxy-propyl)-ammonium bromide and formoterol

At the beginning of each experiment a force of 1.0 g·wt. was applied to the tissues and this was reinstated over a 30 min equilibration period until it remained steady. Tissues were then exposed to 1 μM of the muscarinic agonist, methacholine, to assess tissue viability. Tissues were washed by exchanging the bathing Krebs solution three times. After 30 minutes the tissues were again precontracted with 1 μM methacholine. When the contraction had reached a plateau, 1 nM Formoterol, 10 nM Muscarinic Antagonist (MA2) (Crystalline From A) or a combination of both was added to the bathing media and left for 60 minutes.

Data were collected using the ADInstruments Chart5 for windows software, the tension generated was measured before addition of methacholine and after its response had reached plateau. The response to compound MA2 and/or formoterol was measured at 10 minute intervals following their addition. All responses were expressed as percentage inhibition of the methacholine-induced contraction. The results are depicted in FIG. 6 and Table 2, wherein compound A is (MA2).

TABLE 2
Table 2. % Inhibition of 1 μM Methacholine induced tone by
Formoterol (1 nM), Compound A (10 nM) and Compound A (10 nM)
in the presence of Formoterol (1 nM) in guinea pig trachea in vitro.

| | % inhibition of 1 μM | | | | |
|---|---|---|---|---|---|
| | Methacholine induced tone | | | | |
| Compound | 10 min | 20 min | 30 min | 40 min | 50 min |
| Formoterol (1 nM) | 21 | 37 | 41 | 42 | 44 |
| Compound A (10 nM) | 23 | 62 | 94 | 104 | 106 |
| Compound A (10 nM) in | 55 | 97 | 104 | 105 | 106 |
| the presence of | | | | | |
| Formoterol (1 nM) | | | | | |

### Muscarinic Antagonist 11 (MA11): [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate and formoterol

At the beginning of each experiment a force of 1.0 g·wt. was applied to the tissues and this was reinstated over a 30 min equilibration period until it remained steady. Tissues were then exposed to 1 μM of the muscarinic agonist, methacholine, to assess tissue viability. Tissues were washed by exchanging the bathing Krebs solution three times. After 30 minutes the tissues were again precontracted with 1 μM methacholine. When the contraction had reached a plateau, 1 nM Formoterol, 10 nM Muscarinic Antagonist MA11 or a combination of both was added to the bathing media and left for 60 minutes.

Data were collected using the ADInstruments Chart5 for windows software, the tension generated was measured before addition of methacholine and after its response had reached plateau. The response to compound (MA11) and/or formoterol was measured at 10 minute intervals following their addition. All responses were expressed as percentage inhibition of the methacholine-induced contraction. The results are depicted in FIG. 7 and Table 3, wherein compound B is (MA11).

TABLE 3
Table 3. % Inhibition of 1 μM Methacholine induced tone by
Formoterol (1 nM), Compound B (10 nM) and Compound B (10 nM)
in the presence of Formoterol (1 nM) in guinea pig trachea in vitro.

| | % inhibition of 1 μM | | | | |
|---|---|---|---|---|---|
| | Methacholine induced tone | | | | |
| Compound | 10 min | 20 min | 30 min | 40 min | 50 min |
| Formoterol (1 nM) | 21 | 37 | 41 | 42 | 44 |
| Compound B (10 nM) | 11 | 29 | 49 | 67 | 84 |
| Compound B (10 nM) in | 38 | 80 | 98 | 103 | 107 |
| the presence of | | | | | |
| Formoterol (1 nM) | | | | | |

In Vivo Combination Data

Evaluation of Lung Function in Anaesthetised Guinea Pigs.

Male Dunkin-Hartley guinea pigs (300-600 g) were weighed and dosed with vehicle (0.05M phosphate, 0.1% Tween 80, 0.6% saline, pH 6) or compound via the intratracheal route under recoverable gaseous anaesthesia (5% halothane in oxygen). Animals were dosed with compound or vehicle two hours prior to the administration of methacholine. Guinea pigs were anaesthetised with pentobarbitone (1 mL/kg of 60 mg/mL solution i.p.) approximately 30 minutes prior to the first bronchoconstrictor administration. The trachea was cannulated and the animal ventilated using a constant volume respiratory pump (Harvard Rodent Ventilator model 683) at a rate of 60 breath/min and a tidal volume of 5 mL/kg. A jugular vein was cannulated for the administration of methacholine or maintenance anaesthetic (0.1 mL of pentobarbitone solution, 60 mg/mL, as required).

The animals were transferred to a Flexivent System (SCIREQ, Montreal, Canada) in order to measure airway resistance. The animals were ventilated (quasi-sinusoidal ventilation pattern) at 60 breaths/min at a tidal volume of 5 mL/kg. A positive end expiratory pressure of 2-3 cm H₂O was applied. Respiratory resistance was measured using the Flexivent “snapshot” facility (1 second duration, 1 Hz frequency). Once a stable baseline resistance value had been obtained the animals were given methacholine in ascending doses (0.5, 1, 2, 3 and 5 μg/kg, i.v) at approximately 4-minute intervals via the jugular catheter. After each administration of bronchoconstrictor the peak resistance value was recorded. Guinea pigs were euthanised with approximately 1.0 mL pentobarbitone sodium (Euthatal) intravenously after the completion of the lung function measurements.

Percentage bronchoprotection produced by a compound was calculated at each dose of brochoconstrictor as follows:

$$\% bronchoprotection = \frac{\% {changeR}_{veh} - \% {changeR}_{cmpd}}{\% {changeR}_{veh}}$$

Where % change Rᵥₑₕ is the mean of the maximum percentage change in airway resistance in the vehicle treated group. The results reported were measured after 5 μg/kg methacholine and were expressed as percentage bronchoprotection (mean±s.e.mean).

### β₂-Adrenoceptor Agonist 1: (BA1): N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide and Muscarinic Antagonist 2 (MA2) [2-((R)-Cyclohexyl-hydroxy-phenyl-methyl)-oxazol-5-ylmethyl]-dimethyl-(3-phenoxy-propyl)-ammonium bromide

Guinea pigs were dosed with vehicle, 3 and 27 μg/kg compound (BA1), 0.2 μg/kg compound (MA2) (Crystalline Form A) or a combination of 3 μg/kg Compound (BA1) and 0.2 μg/kg Compound (MA2) via the intratracheal route. Administration of increasing intravenous doses of methacholine (0.5, 1, 2, 3 and 5 μg/kg) evoked dose-related bronchoconstriction in the vehicle treated animals ranging from 13±2.6% at 0.5 μg/kg to 2530±280% at 5 μg/kg two hours after vehicle administration (n=9). Intratracheal administration of Compound (MA2) at 0.2 μg/kg produced 13% inhibition of methacholine-induced bronchoconstriction (2210±268% increase in resistance, n=8). Intratracheal administration of compound (BA1) (3 and 27 μg/kg) 2 hours prior to methacholine produced 17 and 81% inhibition of methacholine-induced bronchoconstriction (2090±239 and 470±221% increase in resistance, respectively; n=8 and 6, respectively). The combination of Compound (MA2) (0.2 μg/kg) and compound (BA1) (3 μg/kg) produced 55% inhibition of methacholine-induced bronchoconstriction (1140±151% increase in resistance; n=8) (see FIG. 8—wherein Compound A is (BA1) and compound Z is (MA2).

### β2-Adrenoceptor Agonist 1: (BA1): N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide and Muscarinic Antagonist 11 (MA11): [2-(4-chloro-benzyloxy)-ethyl]-[2-((R)-cyclohexyl-hydroxy-phenyl-methyl)-oxazol-5-ylmethyl]-dimethyl-ammonium hemi-naphthalene-1,5-disulfonate

Guinea pigs were dosed with vehicle; 1 and 27 μg/kg Compound (BA1), 0.01 μg/kg Compound (MA11) or a combination of 1 μg/kg Compound (BA1) and 0.01 μg/kg Compound (MA11) via the intratracheal route. Administration of increasing intravenous doses of methacholine (0.5, 1, 2, 3 and 5 μg/kg) evoked dose-related bronchoconstriction in the vehicle treated animals ranging from 14±2.6% at 0.5 μg/kg to 2240±269% at 5 μg/kg two hours after vehicle administration (n=10). Intratracheal administration of Compound (MA11) at 0.2 μg/kg produced 16% inhibition of methacholine-induced bronchoconstriction (1880±272% increase in resistance, n=6). Intratracheal administration of Compound (BA1) (1 and 27 μg/kg) 2 hours prior to methacholine produced 38 and 89% inhibition of methacholine-induced bronchoconstriction (1380±333 and 242±69% increase in resistance, respectively; n=8 and 6, respectively). The combination of Compound (MA11) (0.2 μg/kg) and Compound (BA1) (3 μg/kg) produced 43% inhibition of methacholine-induced bronchoconstriction (1273±260% increase in resistance; n=7) (see FIG. 9 wherein Compound A is (BA1) and compound Y is (MA11)).

1. A pharmaceutical product, wherein the product comprises:
a [2-(4-Chloro-benzyloxy)-ethyl]-[2-((R)-Cyclohexyl-hydroxy-phenyl-methyl)-oxazol-5-ylmethyl]-dimethyl-ammonium salt; and
N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

2. (canceled)

3. A product according to claim 1, wherein the [2-(4-chloro-benzyloxy)-ethyl]-[2-((R)-cyclohexyl-hydroxy-phenyl-methyl)-oxazol-5-ylmethyl]-dimethyl-ammonium salt is a bromide salt.

4. A product according to claim 1, wherein the [2-(4-chloro-benzyloxy)-ethyl]-[2-((R)-cyclohexyl-hydroxy-phenyl-methyl)-oxazol-5-ylmethyl]-dimethyl-ammonium salt is a napadisylate salt.

5-7. (canceled)

8. A method according to claim 21, wherein the respiratory disease is chronic obstructive pulmonary disease.

9. A method of treating a respiratory disease, wherein the method comprises simultaneously, sequentially, or separately administering to a patient thereof:
(a) a therapeutically effective dose of a [2-(4-chloro-benzyloxy)-ethyl]-[2-((R)-cyclohexyl-hydroxy-phenyl-methyl)-oxazol-5-ylmethyl]-dimethyl-ammonium salt; and
(b) a therapeutically effective dose of N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

10. A kit, wherein the kit comprises:
a preparation of a [2-(4-chloro-benzyloxy)-ethyl]-[2-((R)-cyclohexyl-hydroxy-phenyl-methyl)-oxazol-5-ylmethyl]-dimethyl-ammonium salt, and
a preparation of N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

11. A pharmaceutical composition comprising, in admixture:
a [2-(4-chloro-benzyloxy)-ethyl]-[2-((R)-Cyclohexyl-hydroxy-phenyl-methyl)-oxazol-5-ylmethyl]-dimethyl-ammonium salt; and
N-[1-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

12. A pharmaceutical product according to claim 1, wherein the product comprises N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide.

13. A pharmaceutical product according to claim 1, wherein the product comprises:
[2-(4-chloro-benzyloxy)-ethyl]-[2-((R)-cyclohexyl-hydroxy-phenyl-methyl)-oxazol-5-ylmethyl]-dimethyl-ammonium napadisylate; and
N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide.

14. A kit according to claim 10, wherein the kit comprises [2-(4-chloro-benzyloxy)-ethyl]-[2-((R)-cyclohexyl-hydroxy-phenyl-methyl)-oxazol-5-ylmethyl]-dimethyl-ammonium napadisylate.

15. A kit according to claim 10, wherein the kit comprises N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide.

16. A kit according to claim 10, wherein the kit comprises:
[2-(4-chloro-benzyloxy)-ethyl]-[2-((R)-cyclohexyl-hydroxy-phenyl-methyl)-oxazol-5-ylmethyl]-dimethyl-ammonium napadisylate; and
N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide.

17. A kit according to claim 10, wherein the kit further comprises instructions for simultaneous administration of the preparations to a patient in need thereof.

18. A kit according to claim 10, wherein the kit further comprises instructions for sequential administration of the preparations to a patient in need thereof.

19. A kit according to claim 10, wherein the kit further comprises instructions for separate administration of the preparations to a patient in need thereof.

20. A pharmaceutical composition according to claim 11, wherein the composition comprises, in admixture:
[2-(4-chloro-benzyloxy)-ethyl]-[2-((R)-cyclohexyl-hydroxy-phenyl-methyl)-oxazol-5-ylmethyl]-dimethyl-ammonium napadisylate; and
N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide.

21. A method according to claim 9, wherein the method comprises simultaneously administering to a patient thereof:
(a) a therapeutically effective dose of a [2-(4-chloro-benzyloxy)-ethyl]-[2-((R)-cyclohexyl-hydroxy-phenyl-methyl)-oxazol-5-ylmethyl]-dimethyl-ammonium salt; and
(b) a therapeutically effective dose of N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

22. A method according to claim 9, wherein the method comprises sequentially administering to a patient thereof:
(a) a therapeutically effective dose of a [2-(4-chloro-benzyloxy)-ethyl]-[2-((R)-cyclohexyl-hydroxy-phenyl-methyl)-oxazol-5-ylmethyl]-dimethyl-ammonium salt; and
(b) a therapeutically effective dose of N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

23. A method according to claim 22, wherein the respiratory disease is chronic obstructive pulmonary disease.

24. A method according to claim 9, wherein the method comprises separately administering to a patient thereof:
(a) a therapeutically effective dose of a [2-(4-chloro-benzyloxy)-ethyl]-[2-((R)-cyclohexyl-hydroxy-phenyl-methyl)-oxazol-5-ylmethyl]-dimethyl-ammonium salt; and
(b) a therapeutically effective dose of N-[2-(diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

25. A method according to claim 24, wherein the respiratory disease is chronic obstructive pulmonary disease.
